(19) United States
(12) Patent Application Publication
(10) Pub. No.: US 2021/0087638 A1
(43) Pub. Date: Mar. 25, 2021

(54) NEXT-GENERATION SEQUENCING ASSAY FOR GENOMIC CHARACTERIZATION AND MINIMAL RESIDUAL DISEASE DETECTION IN THE BONE MARROW, PERIPHERAL BLOOD, AND URINE OF MULTIPLE MYELOMA AND SMOLDERING MYELOMA PATIENTS

(71) Applicant: DANA-FARBER CANCER INSTITUTE, INC., Boston, MA (US)

(72) Inventor: Irene Ghobrial, Boston, MA (US)

(73) Assignee: DANA-FARBER CANCER INSTITUTE, INC., Boston, MA (US)

(21) Appl. No.: 17/029,684

(22) Filed: Sep. 23, 2020

Related U.S. Application Data
(60) Provisional application No. 62/904,532, filed on Sep. 23, 2019.

(51) Int. Cl. C12Q 1/6886

(52) U.S. Cl. CPC C12Q 1/6886; C12Q 2600/156; C12Q 2600/106; C12Q 2600/118

(57) ABSTRACT

The present invention relates to methods for the personalized detection of Minimal Residual Disease (MRD) from the peripheral blood, urine, or bone marrows through patient-specific translocation breakpoints and VDJ rearrangements, as well as copy number alterations (CNAs) and single nucleotide variants (SNV) specific to Multiple myeloma (MM).

### RELATED APPLICATIONS

This application claims the benefit of priority under 35 U.S.C. § 119(e) to U.S. Provisional Application No. 62/904,532, filed Sep. 23, 2019, which is incorporated herein by reference in its entirety.

### BACKGROUND OF THE INVENTION

Multiple myeloma (MM) is an incurable plasma cell malignancy, characterized by marked genetic heterogeneity that relapses in most patients. Prior to the invention described herein, there was a pressing need to identify methods to identify high-risk smoldering multiple myeloma patients based on their genomic profile and to monitor response to treatment by detecting Minimal Residual Disease (MRD) for early detection of relapse.

### SUMMARY OF THE INVENTION

The invention is based, at least in part, upon the personalized detection of multiple myeloma (MM) specific copy number alterations (CNAs) and single nucleotide variants (SNVs) as well as Minimal Residual Disease (MRD) from the peripheral blood, urine, or bone marrow of a patient.

Described herein are methods of individualized monitoring of response to treatment for detection of Minimal Residual Disease (MRD) in blood or urine samples of Multiple Myleoma (MM) patients and disease progression in MM, smoldering multiple myeloma (SMM) and monoclonal gammopathy of undetermined significance (MGUS), for which there is growing need in the field, given MM's marked genetic heterogeneity and tendency to relapse.

Also described herein are methods for two one-size-fits-all assays for CNAs, SNVs, translocations, and VDJ rearrangement detection in MM and other B-cell malignancies, a well-benchmarked short-read assay for affordable Deep Targeted Sequencing (DTS) and a targeted long-read assay that will allow for improved translocation and VDJ rearrangement detection, as well as confident identification of somatic hypermutation.

Methods of determining whether a subject, e.g., a human subject, with monoclonal gammopathy of undetermined significance (MGUS) or smoldering multiple myeloma (SMM) will progress to multiple myeloma (MM) in a subject are carried out by obtaining a test sample from a subject having MGUS, SMM, or at risk of developing MM; detecting a somatic aberration in at least one MM-associated gene in the test sample as compared to the MM-associated gene in a reference sample; and determining that the subject will progress to MM.

For example, the at least one MRD-associated gene comprises at least one of Actin Gamma 1 (ACTG1), Protein kinase B (AKT1), Anaplastic Lymphoma Kinase (ALK), AT Rich Interactive Domain 1A (ARID1A), ASXL Transcriptional Regulator 1 (ASXL1), ASXL Transcriptional Regulator 3 (ASXL3), Ataxia-Telangiectasia Mutated (ATM), Ataxia telangiectasia and Rad3 related (ATR), alpha-thalassemia/mental retardation, X-linked (ATRX), B-cell CLL/lymphoma 7 (BCL7A), B-Raf Proto-Oncogene, Serine/Threonine Kinase (BRAF), Cyclin D1 (CCND1), Cadherin-4 (CDH4), Cyclin-dependent kinase inhibitor 1B (CDKN1B), Cyclin Dependent Kinase Inhibitor 2C (CDKN2C), CREB-binding protein (CREBBP), Chr. C-X-C chemokine receptor type 4 (CXCR4), CYLD lysine 63 deubiquitinase (CYLD), Exosome complex exonuclease RRP44 (DIS3), DNA Methyltransferase 3 Alpha (DNMT3A), Early growth response protein 1 (EGR1), E1A binding protein p300 (EP300), ETS translocation variant 4 (ETTZ4), Protein FAM46C (FAM46C), Fibroblast growth factor receptor 3 (FGFR3), Far Upstream Element Binding Protein 1 (FUBP1), HIST1H1C, HIST1H1E, HIST1H3G, HIST1H3H, Isocitrate Dehydrogenase 1 (IDH1), Isocitrate Dehydrogenase 2 (IDH2), Insulin-like Growth Factor 1 Receptor (IGF1R), Interferon Regulatory Factor 4 (IRF4), Lysine-Specific Demethylase 5C (KDM5C), Lysine-specific Demethylase 6A (KDM6A), Histone-lysine N-methyltransferase 2A (KMT2A), Lysine Methyltransferase 2B (KMT2B), Lysine Methyltransferase 2C (KMT2C), Lysine Methyltransferase 2D (KMT2D), Kirsten Rat Sarcoma (KRAS), Lymphotoxin-beta (LTB), MAF, MAFB, myc-associated factor X (MAX), Myeloid Differentiation Primary Response Protein (MYD88), Nuclear Receptor Corepressor 1 (NCOR1), Neurofibromin 1 (NF1), Nuclear Factor Of Kappa Light Polypeptide Gene Enhancer In B-Cells Inhibitor (NFKBIA), Neurogenic Locus Notch Homolog Protein 1 (NOTCH1), Neuroblastoma RAS (NRAS), NRM, Phosphatidylinositol-4,5-Bisphosphate 3-Kinase Catalytic Subunit Alpha (PIK3CA), Protein Phosphatase, Mg2+/Mn2+Dependent 1D (PPM1D), PRAME Family Member 2 (PRAMEF2), PR Domain Zinc Finger Protein 1 (PRDM1), Serine/threonine-Protein Kinase D2 (PRKD2), Prune Homolog 2 With BCH Domain (PRUNE2), Protein-Tyrosine Phosphatase Non-Receptor Type 11 (PTPN11), RAS P21 Protein Activator 2 (RASA2), Retinoblastoma Associated Protein (RB1), SET Domain Containing 2, Histone Lysine Methyltransferase (SETD2), Splicing Factor 3b Subunit 1 (SF3B1), SP140, Ten-Eleven Translocation Methylcytosine Dioxygenase 2 (TET2), TDP-Glucose 4,6-Dehydratase (TGDS), Tumor Protein p53 (TP53), TNF Receptor Associated Factor 3 (TRAF3), and Zinc Finger Homeobox Protein 3 (ZFHX3). In some cases, the MRD-associate gene comprises each of the genes listed above. In one aspect, the at least one MRD-associated gene comprises KRAS and NRAS. In another aspect, the at least one MRD-associated gene comprises TP53 and ATM. In yet another aspect, the at least one MRD-associated gene comprises an MYC oncogene.
Exemplary somatic aberrations include a single nucleotide variation (SNV), a copy number alteration (CNA), a chromosome translocation breakpoint, or a variable (V), diversity (D), and joining (J; VDJ) rearrangement.

Suitable samples include those obtained from blood, urine, or bone marrow. In some cases, the sample comprises cell free deoxyribonucleic acid (cfDNA) or circulating tumor cells (CTCs). For example, the reference sample is obtained from a healthy normal control sample, a MGUS sample, an SMM sample, or an MM sample. The reference sample is from one individual or an aggregate of more than one individual, e.g., from a publicly-accessible database.

In some cases, the somatic aberration of the MM-associated gene is detected via next generation sequencing (NGS), whole exome sequencing (WES), or deep targeted sequencing (DTS).

Preferably, the method further comprises treating the subject with a chemotherapeutic agent, radiation therapy, a corticosteroid, a bone marrow transplant, or a stem cell transplant. For example, the chemotherapeutic agent comprises elotuzumab, lenalidomide, dexamethasone, melphlan, vincristine, doxorubicin, etoposide, bendamustine, or cyclophosphamide.

In one aspect, the method is repeated over time, wherein an increase in somatic alteration of the MM-associated gene over time indicates a corresponding increase in progression of MM. Also provided are methods of determining whether a subject with minimal residual disease (MRD) will relapse to MM in a subject comprising: obtaining a test sample from a subject having MRD; detecting a somatic aberration in at least one MM-associated gene in the test sample as compared to the MM-associated gene in a reference sample; and determining that the subject will relapse to MM.

Preferably, the methods further comprise treating the subject with a chemotherapeutic agent, radiation therapy, a corticosteroid, a bone marrow transplant, or a stem cell transplant. Exemplary samples are obtained from blood, urine, or bone marrow.

Methods of monitoring therapeutic efficacy of treatment in a subject with MM are carried out by administering treatment to the subject having MM; obtaining a test sample from the subject; detecting a somatic aberration in at least one MM-associated gene in the test sample as compared to the MM-associated gene in a reference sample.

It is determined that the treatment in the subject is not effective if the level of the somatic aberrations in the test sample is higher as compared to the level of somatic aberration in the reference sample, and the treatment is modified. It is determined that the treatment in the subject is effective if the level of the somatic aberrations in the test sample is lower than the level of somatic aberration in the reference sample.

For example, the treatment comprises administration of a chemotherapeutic agent, radiation therapy, corticosteroids, a bone marrow transplant, or a stem cell transplant.

In some cases, the method is repeated over time. It is determined that the treatment is effective if the level of the somatic aberration is lower over time. It is determined that the treatment is ineffective if the level of somatic aberration is the same or higher over time.

### Definitions

As used herein, “obtaining” as in “obtaining a sample” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The term “progression,” is defined herein as the prediction of the degree of severity of the MRRD and of its evolution as well as the prospect of recovery as anticipated from usual course of the disease. Once the aggressiveness has been determined, appropriate methods of treatments are chosen.

The term “sample” as used herein refers to a biological sample obtained for the purpose of evaluation in vitro. Exemplary tissue samples for the methods described herein include tissue samples from patients diagnosed with multiple myeloma and/or MRD. With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma or serum). Preferred samples are whole blood, serum, plasma, bone marrow, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from, e.g., Multiple Myeloma or MRD is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to affect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

Any compositions or methods provided herein can be combined with one or more of any of the other compositions and methods provided herein.

Where applicable or not specifically disclaimed, any one of the embodiments described herein are contemplated to be able to combine with any other one or more embodiments, even though the embodiments are described under different aspects of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-1B, FIG. 2A-2C, FIG. 3, FIG. 4A-4B were obtained from S. Manier, Whole-exome sequencing of cell-free DNA and circulating tumor cells in multiple myeloma. Nature Communications, 9:11 (2018), and is incorporated herein by reference.

FIG. 1A-FIG. 1B shows detectability and clinical correction of CTCs and cfDNA in multiple myeloma. FIG. 1A is a series of graphs showing ULP-WGS tumor fraction estimates for 107 cfDNA samples and 56 CTC samples from MGUS, SMM, MM, and relapse patients; 58%, 28%, and 17% of cfDNA samples had ≥0.03, 0.05, and 0.1 tumor fractions, respectively (blue); 96%, 50%, and 21% of CTC samples had ≥0.03, 0.05, and 0.1 tumor fractions, respectively (green). FIG. 1B is a series of box and whisker plots showing Tumor fractions of cfDNA samples (blue) and CTC number×tumor fractions 4 of CTC samples (green) correlate with clinical stage of MM patients. The comparisons of the tumor fraction in cfDNA and CTC among different disease status (MGUS, SMM, newly diagnosed MM, and relapse) were performed by using Kruskal-Wallis test (p-value<0.001 for cfDNA; p-value=0.001 for CTC). The distributions of tumor fraction (%) in each group are shown as boxplots, where the central rectangle spans the first to the third quartile (interquartile range or IQR). A segment inside the rectangle shows the median, and whiskers above and below the box show the value 1.5 IQR above or below the third or the first quartile, respectively

FIG. 2A-2C shows cfDNA can be used to monitor disease progression. FIG. 2A is a schematic and graph showing that progression monitoring or response to therapy can be done with liquid biopsy. Comparison of copy ratios between cfDNA ULP-WGS from sequential samples from same MM patients. Tumor fraction (pink) and free light chain ratio (green) for each patient are also shown in this figure. Amplification (red), deletion (green), and copy neutral (blue) are indicated. FIG. 2B is a schematic and graph showing progression over a period of 2 months after the therapy. The tumor fraction was increased from 11% to 46% and was correlated with serum free light chain assay (immunoglobulin light chains that are circulating in serum in a free state are called free light chains and measuring the serum level of FLCs is a clinical blood test). FIG. 2C is a schematic and graph showing a very good partial response to therapy and decreased tumor fraction as well as serum free light chain assay. This data highlights that simple blood draw can be used to monitor disease progression and therapy response instead of invasive biopsies. This is especially important for patients with precursor conditions of MM who will have to be monitored for a while.

FIG. 3 shows whole-exome sequencing of cfDNA, CTCs, and Bone Marrow (BM) tumor samples. FIG. 3 is a series of color plots showing Presence of clonal (navyblue) and subclonal (yellow) somatic mutations in BM, cfDNA, and CTC WES. Snow color represents mutations that were not detected with ≥0.9 detection power and gray color represents mutation sites with <0.9 detection power. MM and actionable pan-cancer related genes and purity of each sample are indicated.

FIG. 4A-4B shows somatic mutations and copy number alterations in matched cfDNA, CTCs, and BM tumor samples. FIG. 4A is a color plot showing the alteration status of MM and actionable pan-cancer mutations and focal SCNAs are shown for bone marrow biopsies, cfDNA, and CTC samples from same MM patients. Hotspot mutation (black), missense mutation (green), nonsense mutation (purple), gain (red), and loss (blue) are indicated for specific SSNVs and SCNAs. Mutations that were not detected with ≥0.9 detection power (snow) and mutations with <0.9 detection power (gray) are distinguished in this figure. FIG. 4B is a color plot showing the alteration status of MM and actionable pan-cancer mutations and focal SCNAs are shown for bone marrow biopsies and cfDNA samples from same MM patients. Hotspot mutation (black), missense mutation (green), nonsense mutation (purple), gain (red), and loss (blue) were indicated for specific SSNVs and SCNAs. Mutations that were not detected with ≥0.9 detection power (snow) and mutations with <0.9 detection power (gray) are distinguished in this figure. Mutations were predicted using Mutect and SCNAs were predicted using ReCapSeg.

FIG. 5 is a schematic showing leveraging duplex sequencing for error suppression.

FIG. 6 is a series of box and whisker plots showing that duplex sequencing affords 1,000-fold fewer errors than regular NGS.

FIG. 7 is a graph showing the myeloma-specific gene panel performs well with high % enrichment of selected bases.

FIG. 8 is a graph showing the performance of the 69-gene panel compared to a 400 pan cancer gene panel in terms of median target coverage (MTC). The 69-gene panel show higher MTC based on testing 12 normal and tumor samples.

FIG. 9A-9D is a series of Kaplan-Meier curves and a series of Forest plots showing the analysis of time to progression in SMM patients. FIG. 9A is a Kaplan-Meier curve showing the analysis of time to progression in SMM patients with MAPK pathway mutations (KRAS and NRAS). FIG. 9B is a Kaplan-Meier curve showing the analysis of time to progression in SMM with MYC alterations, including translocation and amplifications. FIG. 9C is a Kaplan-Meier curve for analysis of time to progression in SMM patients with DNA repair pathway alterations (deletion 17p, TP53 and ATM SNVs). FIG. 9D is a series of Forest plots of multivariate cox-regression of the high-risk genomic alterations and the clinical risk model.

FIG. 10 is a table showing the performance of the clinical models with and without the genetic model. Improvement in goodness of fit was assessed with a likelihood ratio test. The genetic model significantly improved the fit of the clinical-only models. A global assessment of each model was also assessed using a C-statistic for censored survival data. The statistic for each time-to-event model is reported with a 95% confidence interval. Values range between 0.5 to 1 indicating a poor to perfect model.

FIG. 11 is a table showing a cohort overview of SMM patients.

FIG. 12 is a schematic and series of graphs showing cfDNA sorted by EOT response from best to worst.

FIG. 13 is a schematic and series of graphs showing gDNA sorted by EOT response from best to worst.

FIG. 14 is a schematic showing the design of mutational panel for finger printing and MRD detection.

### DETAILED DESCRIPTION OF THE INVENTION

The invention is based, at least in part, upon the personalized detection of patient-specific copy number alterations (CNAs) and single nucleotide variants (SNV) specific to Multiple myeloma (MM) in two different settings: (1) precursor conditions of MM, namely Smoldering multiple myeloma (SMM), and (2) for Minimal Residual Disease (MRD) detection in the peripheral blood, urine, or bone marrow.

Described herein is the use of cfDNA to monitor patients with SMMs (50 patient samples at baseline). Also described herein is the use of cdDNA as a tool for MRD testing (50 patient samples at 5 time points).

This invention represents an improvement over currently available methods which do not tailor baitset design on individual patients' alterations, do not use Unique Molecular Identifiers (UMIs) to correct for Polymerase Chain Reaction (PCR)-induced duplicates, and do not capture SNVs or CNAs through Deep Targeted Sequencing. Personalizing baitset design is very important for cancers like Multiple Myeloma (MM), which are so markedly heterogeneous. The approach ensures that the major alterations of each patient are followed efficiently over time, including VDJ rearrangement sequence, CNAs and translocations that are quite challenging to capture by regular Targeted Sequencing. The ability to follow those through UMI-corrected Targeted sequencing efficiently and with confidence at very high depth of coverage keeps costs down.

Multiple myeloma (MM) is an incurable plasma cell malignancy, characterized by marked genetic heterogeneity and multiple relapses in most patients. It is almost always preceded by asymptomatic precursor stages, namely monoclonal gammopathy of undetermined significance (MGUS) and smoldering multiple myeloma (SMM) (1, 2). SMM patients have a higher risk of progression to MM (10%/year), compared to MGUS (1%/year) (3), although some patients progress rapidly, others remain in an MGUS-like state for years. As such, a method to detect MM-specific genomic alterations in blood and tissue samples of SMM patients is needed to identify those at a high risk of disease progression to overt MM. It was recently reported that alterations of the MAPK pathway (KRAS and NRAS SNVs), the DNA repair pathway (deletion 17p, TP53 and ATM SNVs), and MYC (translocations or CNVs) were all independent risk factors of progression and considered high-risk genomic biomarkers after accounting for clinical risk staging (4). This panel is considered a companion to this new genomic score that identifies high-risk patients who need therapeutic intervention.

Moreover, this technology may be used to follow response to treatment and identify Minimal Residual Disease (MRD) for early detection of relapse that can lead to better outcomes for patients. As multiple myeloma resides in the bone marrow, sequential samples that would allow for such tumor burden monitoring require serial bone marrow biopsies, which are painful procedures and inconvenient.

A method of MRD VDJ rearrangement detection with a targeted amplicon sequencing approach (Adaptive Biosciences) has already been approved by the FDA and is used as the standard of care, although it is only applicable in genomic DNA from bone marrow samples. Also, the FDA recently allowed the use of MRD as an endpoint for clinical trials in newly diagnosed patients, indicating that this test can be used for endpoints of large clinical trials and clinical management.

Described herein is a method that can be used in the peripheral blood or urine, as well as the bone marrow, and utilizes patient-specific, copy number alterations (CNAs) and single nucleotide variants (SNV) specific to MM, allowing detection of MRD in a personalized way. The methods also allow for tracking of clonal progression and characterization of the genetic profile at every timepoint. The ability to characterize the emerging clones' genetic profile at relapse is of great importance, as it can further inform clinical management and treatment (precision therapy).

Accordingly, the methods described herein are useful for MRD testing, as well as tracking and characterizing disease progression in patients under therapy, but also asymptomatic patients under observation, whose genetic profile can lead to changes in management. The ability to perform this assay on peripheral blood (cfDNA & CTCs) or urine samples is particularly important, as access to such samples is easier and less risky, such that the course of disease progression can be followed up much more regularly. For a summary of the innovations involved, see below.

Cell-free DNA largely consists of normal DNA fragments and thus a method to estimate the percentage of tumor DNA in that pool is required. Ultra-Low-Pass Whole-Genome Sequencing (ULP-WGS) of cfDNA involves affordable, low-depth (0.5×) sequencing of the genome that is sufficient to call copy-number alterations (CNAs), identify the presence of tumor DNA and estimate tumor fraction, which in turn can be used to triage samples appropriately. Analysis of the circulating cell-free methylated DNA (cf-methylDNA) using previously described methylome sequencing techniques can provide an alternative way of detecting the presence of tumor in the samples and estimating tumor purity.

However, significant depth of coverage is required for detection of genetic alterations. Deep Targeted Sequencing (DTS) can provide that, helping identify tumor cells with high sensitivity and, in serial samples, giving an overview of changes in the tumor's genomic landscape that might underlie resistance to treatment and disease progression. The combination of ULP-WGS or cf-methylome sequencing and DTS of cfDNA/CTCs from blood or urine is thus a cost-effective way of following patients' response to treatment and disease progression.

As described herein, to address the issue of Polymerase Chain Reaction (PCR)-induced duplicate reads, an inherent limitation of library preparation for sequencing that reduces its sensitivity, Unique Molecular Identifiers (UMIs) were incorporated in the DNA library preparation, which tag each original DNA fragment before PCR amplification, allowing for more accurate estimation of the number of reads from a particular region and increasing the method's sensitivity for detection of genetic alterations. A UMI-DTS baitset was developed, targeting a curated list of 63 genes commonly mutated in MM, as described in Lohr et al., Bolli et al., Walker et al., and the Multiple Myeloma Research Foundation's (MMRF) database, as well as 32 genes involved in Clonal Hematopoiesis of Indeterminate Potential (CHIP).

Using in silico analysis and the portals of TCGA, CBioportal, and Polyphen, long genes (ZFHX4, EGR1, and HUWE1) that are known to have high background mutation rate, and thus false positive mutations, were filtered out of a list of 95 manually-curated genes from both sets. Non-deleterious common germline variants in the 63 genes were also filtered out to avoid reporting those genes and their single nucleotide variants as true mutations. The analysis led to the identification of 69 genes and their specific exons that frequently occur in MM and CHIP in more than 5% of patients (Table 1)

TABLE 1

| TARGET LEVEL INFO | | | | | | Gene Level | |
|---|---|---|---|---|---|---|---|
| Chromosome | Start | End | Size | Gene | Gene2 | ACTG1 | Alias |
| 17 | 79477603 | 79478709 | 1107 | NM_001614 | ACTG1 | AKT1 | |
| 17 | 79478852 | 79479445 | 594 | NM_001614 | ACTG1 | ALK | |
| 14 | 105246425 | 105246553 | 129 | NM_005163 | AKT1 | ARID1A | |
| 14 | 105239792 | 105239917 | 126 | NM_005163 | AKT1 | ASXL1 | |
| 2 | 29456431 | 29456562 | 132 | NM_004304 | ALK | ASXL3 | |
| 2 | 29445383 | 29445473 | 91 | NM_004304 | ALK | ATM | |
| 2 | 29443572 | 29443701 | 130 | NM_004304 | ALK | ATR | |
| 2 | 29432652 | 29432744 | 93 | NM_004304 | ALK | ATRX | |
| 1 | 27022864 | 27022985 | 122 | NM_139135 | ARID1A | BCL7A | |
| 1 | 27023022 | 27024072 | 1051 | NM_139135 | ARID1A | BRAF | |
| 1 | 27056101 | 27056480 | 380 | NM_139135 | ARID1A | CCND1 | |
| 1 | 27057585 | 27058235 | 651 | NM_139135 | ARID1A | CDH4 | |
| 1 | 27059053 | 27059377 | 325 | NM_139135 | ARID1A | CDKN1B | |
| 1 | 27087301 | 27087726 | 426 | NM_139135 | ARID1A | CDKN2C | |
| 1 | 27087761 | 27088107 | 347 | NM_139135 | ARID1A | CREBBP | |
| 1 | 27088588 | 27088822 | 235 | NM_139135 | ARID1A | CXCR4 | |
| 1 | 27089349 | 27089863 | 515 | NM_139135 | ARID1A | CYLD | |
| 1 | 27092621 | 27093097 | 477 | NM_139135 | ARID1A | DIS3 | |
| 1 | 27094254 | 27094629 | 376 | NM_139135 | ARID1A | DNMT3A | |
| 1 | 27097595 | 27097913 | 319 | NM_139135 | ARID1A | EGR1 | |
| 1 | 27098862 | 27099136 | 275 | NM_139135 | ARID1A | EP300 | |
| 1 | 27099223 | 27099522 | 300 | NM_139135 | ARID1A | ETV4 | |
| 1 | 27099699 | 27100530 | 832 | NM_139135 | ARID1A | FAM46C | |
| 1 | 27100764 | 27101809 | 1046 | NM_139135 | ARID1A | FGFR3 | |
| 1 | 27102033 | 27102434 | 402 | NM_139135 | ARID1A | FUBP1 | |
| 1 | 27105371 | 27107412 | 2042 | NM_139135 | ARID1A | HIST1H1C | |
| 20 | 31021073 | 31025153 | 4081 | ASXL1_E11-12 | ASXL1 | HIST1H1E | |
| 2 | 25964881 | 25967361 | 2481 | ASXL2 | ASXL2 | HIST1H3G | |
| 2 | 25972543 | 25973303 | 761 | ASXL2 | ASXL2 | HIST1H3H | |
| 2 | 25976335 | 25976575 | 241 | ASXL2 | ASXL2 | IDH1 | |
| 2 | 25978834 | 25979034 | 201 | ASXL2 | ASXL2 | IDH2 | |
| 2 | 25982292 | 25982572 | 281 | ASXL2 | ASXL2 | IGF1R | |
| 2 | 25990383 | 25990663 | 281 | ASXL2 | ASXL2 | IRF4 | |
| 2 | 25991518 | 25991718 | 201 | ASXL2 | ASXL2 | KDM5C | |
| 2 | 25991749 | 25991909 | 161 | ASXL2 | ASXL2 | KDM6A | |
| 2 | 25994238 | 25994478 | 241 | ASXL2 | ASXL2 | KMT2A | MLL |
| 2 | 26022188 | 26022468 | 281 | ASXL2 | ASXL2 | KMT2B | |
| 2 | 26029033 | 26029273 | 241 | ASXL2 | ASXL2 | KMT2C | |
| 2 | 26058307 | 26058507 | 201 | ASXL2 | ASXL2 | KMT2D | |
| 2 | 26068333 | 26068453 | 121 | ASXL2 | ASXL2 | KRAS | |
| 2 | 26100982 | 26101142 | 161 | ASXL2 | ASXL2 | LTB | |
| 18 | 31158596 | 31158649 | 54 | ASXL3 | ASXL3 | MAF | |
| 18 | 31187558 | 31187640 | 83 | ASXL3 | ASXL3 | MAFB | |
| 18 | 31314274 | 31314379 | 106 | ASXL3 | ASXL3 | MAX | |
| 18 | 31318451 | 31320407 | 1957 | ASXL3 | ASXL3 | MYD88 | |
| 18 | 31322852 | 31326652 | 3801 | ASXL3 | ASXL3 | NCOR1 | |
| 11 | 108098291 | 108098658 | 368 | ATM_E3 | ATM | NF1 | |
| 11 | 108099857 | 108100097 | 241 | ATM_E5 | ATM | NFKBIA | |
| 11 | 108106338 | 108106636 | 299 | ATM_E6 | ATM | NOTCH1 | |
| 11 | 108114644 | 108114939 | 296 | ATM_E7 | ATM | NRAS | |
| 11 | 108115468 | 108115838 | 371 | ATM_E8 | ATM | NRM | |
| 11 | 108117632 | 108117925 | 294 | ATM_E9 | ATM | PIK3CA | |
| 11 | 108119617 | 108119897 | 281 | ATM_E10 | ATM | PPM1D | |
| 11 | 108121397 | 108121815 | 419 | ATM_E11 | ATM | PRAMEF2 | |
| 11 | 108122509 | 108122845 | 337 | ATM_E12 | ATM | PRDM1 | |
| 11 | 108123491 | 108123723 | 233 | ATM_E13 | ATM | PRKD2 | |
| 11 | 108124528 | 108124778 | 251 | ATM_E14 | ATM | PRUNE2 | |
| 11 | 108126907 | 108127147 | 241 | ATM_E15 | ATM | PTPN11 | |
| 11 | 108128118 | 108128358 | 241 | ATM_E16 | ATM | RASA2 | |
| 11 | 108129630 | 108129857 | 228 | ATM_E17 | ATM | RB1 | |
| 11 | 108137837 | 108138143 | 307 | ATM_E18 | ATM | SETD2 | |
| 11 | 108139087 | 108139376 | 290 | ATM_E19 | ATM | SF3B1 | |
| 11 | 108141694 | 108142175 | 482 | ATM_E20 | ATM | SP140 | |
| 11 | 108143216 | 108143637 | 422 | ATM_E22 | ATM | TET2 | |
| 11 | 108150170 | 108150394 | 225 | ATM_E24 | ATM | TGDS | |
| 11 | 108151637 | 108151948 | 312 | ATM_E25 | ATM | TP53 | |
| 11 | 108153381 | 108153746 | 366 | ATM_E26 | ATM | TRAF3 | |
| 11 | 108154918 | 108155216 | 299 | ATM_E27 | ATM | ZFHX3 | |
| 11 | 108158295 | 108158495 | 201 | ATM_E28 | ATM | | |
| 11 | 108159630 | 108159886 | 257 | ATM_E29 | ATM | | |
| 11 | 108160279 | 108160575 | 297 | ATM_E30 | ATM | | |
| 11 | 108163292 | 108163572 | 281 | ATM_E31 | ATM | | |
| 11 | 108164019 | 108164299 | 281 | ATM_E32 | ATM | | |
| 11 | 108165599 | 108165879 | 281 | ATM_E33 | ATM | | |
| 11 | 108167961 | 108168161 | 201 | ATM_E34 | ATM | | |
| 11 | 108170386 | 108170666 | 281 | ATM_E35 | ATM | | |
| 11 | 108172325 | 108172591 | 267 | ATM_E36 | ATM | | |
| 11 | 108173513 | 108173807 | 295 | ATM_E37 | ATM | | |
| 11 | 108175350 | 108175630 | 281 | ATM_E38 | ATM | | |
| 11 | 108178504 | 108178767 | 264 | ATM_E39 | ATM | | |
| 11 | 108180841 | 108181084 | 244 | ATM_E40 | ATM | | |
| 11 | 108183078 | 108183281 | 204 | ATM_E41 | ATM | | |
| 11 | 108186493 | 108186895 | 403 | ATM_E42 | ATM | | |
| 11 | 108188036 | 108188293 | 258 | ATM_E44 | ATM | | |
| 11 | 108190630 | 108190945 | 316 | ATM_E45 | ATM | | |
| 11 | 108191987 | 108192209 | 223 | ATM_E46 | ATM | | |
| 11 | 108195988 | 108196299 | 312 | ATM_E47 | ATM | | |
| 11 | 108196728 | 108197008 | 281 | ATM_E48 | ATM | | |
| 11 | 108198328 | 108198530 | 203 | ATM_E49 | ATM | | |
| 11 | 108199728 | 108200015 | 288 | ATM_E50 | ATM | | |
| 11 | 108200900 | 108201186 | 287 | ATM_E51 | ATM | | |
| 11 | 108202127 | 108202327 | 201 | ATM_E52 | ATM | | |
| 11 | 108202560 | 108202804 | 245 | ATM_E53 | ATM | | |
| 11 | 108203431 | 108203677 | 247 | ATM_E54 | ATM | | |
| 11 | 108204491 | 108204753 | 263 | ATM_E55 | ATM | | |
| 11 | 108205645 | 108206023 | 379 | ATM_E56 | ATM | | |
| 11 | 108206486 | 108206766 | 281 | ATM_E57 | ATM | | |
| 11 | 108213903 | 108214169 | 267 | ATM_E58 | ATM | | |
| 11 | 108216412 | 108216692 | 281 | ATM_E59 | ATM | | |
| 11 | 108217914 | 108218148 | 235 | ATM_E60 | ATM | | |
| 11 | 108224449 | 108224669 | 221 | ATM_E61 | ATM | | |
| 11 | 108225486 | 108225746 | 261 | ATM_E62 | ATM | | |
| 11 | 108235756 | 108236283 | 528 | ATM_E63 | ATM | | |
| 3 | 142168214 | 142168461 | 248 | NM_001184 | ATR | | |
| 3 | 142169349 | 142169582 | 234 | NM_001184 | ATR | | |
| 3 | 142171839 | 142172118 | 280 | NM_001184 | ATR | | |
| 3 | 142176426 | 142176607 | 182 | NM_001184 | ATR | | |
| 3 | 142177767 | 142177987 | 221 | NM_001184 | ATR | | |
| 3 | 142177992 | 142178242 | 251 | NM_001184 | ATR | | |
| 3 | 142180741 | 142180936 | 196 | NM_001184 | ATR | | |
| 3 | 142183917 | 142184096 | 180 | NM_001184 | ATR | | |
| 3 | 142185142 | 142185397 | 256 | NM_001184 | ATR | | |
| 3 | 142186732 | 142186944 | 213 | NM_001184 | ATR | | |
| 3 | 142188157 | 142188463 | 307 | NM_001184 | ATR | | |
| 3 | 142188829 | 142189063 | 235 | NM_001184 | ATR | | |
| 3 | 142203938 | 142204193 | 256 | NM_001184 | ATR | | |
| 3 | 142211934 | 142212213 | 280 | NM_001184 | ATR | | |
| 3 | 142215163 | 142215394 | 232 | NM_001184 | ATR | | |
| 3 | 142215820 | 142216079 | 260 | NM_001184 | ATR | | |
| 3 | 142217428 | 142217674 | 247 | NM_001184 | ATR | | |
| 3 | 142217994 | 142218116 | 123 | NM_001184 | ATR | | |
| 3 | 142218413 | 142218586 | 174 | NM_001184 | ATR | | |
| 3 | 142222151 | 142222332 | 182 | NM_001184 | ATR | | |
| 3 | 142223937 | 142224157 | 221 | NM_001184 | ATR | | |
| 3 | 142226736 | 142226990 | 255 | NM_001184 | ATR | | |
| 3 | 142231079 | 142231348 | 270 | NM_001184 | ATR | | |
| 3 | 142232295 | 142232527 | 233 | NM_001184 | ATR | | |
| 3 | 142234158 | 142234489 | 332 | NM_001184 | ATR | | |
| 3 | 142238489 | 142238677 | 189 | NM_001184 | ATR | | |
| 3 | 142241523 | 142241729 | 207 | NM_001184 | ATR | | |
| 3 | 142242791 | 142243090 | 300 | NM_001184 | ATR | | |
| 3 | 142253866 | 142254113 | 248 | NM_001184 | ATR | | |
| 3 | 142254887 | 142255067 | 181 | NM_001184 | ATR | | |
| 3 | 142257278 | 142257505 | 228 | NM_001184 | ATR | | |
| 3 | 142259702 | 142259972 | 271 | NM_001184 | ATR | | |
| 3 | 142261429 | 142261643 | 215 | NM_001184 | ATR | | |
| 3 | 142266512 | 142266791 | 280 | NM_001184 | ATR | | |
| 3 | 142268306 | 142268562 | 257 | NM_001184 | ATR | | |
| 3 | 142268933 | 142269169 | 237 | NM_001184 | ATR | | |
| 3 | 142272054 | 142272245 | 192 | NM_001184 | ATR | | |
| 3 | 142272430 | 142272867 | 438 | NM_001184 | ATR | | |
| 3 | 142274663 | 142275019 | 357 | NM_001184 | ATR | | |
| 3 | 142275147 | 142275436 | 290 | NM_001184 | ATR | | |
| 3 | 142277431 | 142277665 | 235 | NM_001184 | ATR | | |
| 3 | 142278070 | 142278303 | 234 | NM_001184 | ATR | | |
| 3 | 142279053 | 142279320 | 268 | NM_001184 | ATR | | |
| 3 | 142280069 | 142280299 | 231 | NM_001184 | ATR | | |
| 3 | 142281049 | 142282002 | 954 | NM_001184 | ATR | | |
| 3 | 142284922 | 142285153 | 232 | NM_001184 | ATR | | |
| 3 | 142286887 | 142287052 | 166 | NM_001184 | ATR | | |
| 3 | 142297420 | 142297621 | 202 | NM_001184 | ATR | | |
| x | 76763807 | 76764127 | 321 | ATRX_E35 | ATRX | | |
| x | 76776209 | 76776449 | 241 | ATRX_E34 | ATRX | | |
| x | 76776828 | 76777028 | 201 | ATRX_E33 | ATRX | | |
| x | 76777683 | 76777923 | 241 | ATRX_E32 | ATRX | | |
| x | 76778664 | 76778944 | 281 | ATRX_E31 | ATRX | | |
| x | 76812898 | 76813138 | 241 | ATRX_E30 | ATRX | | |
| x | 76814088 | 76814368 | 281 | ATRX_E29 | ATRX | | |
| x | 76829665 | 76829905 | 241 | ATRX_E28 | ATRX | | |
| x | 76845256 | 76845456 | 201 | ATRX_E27 | ATRX | | |
| x | 76849102 | 76849382 | 281 | ATRX_E26 | ATRX | | |
| x | 76854790 | 76855070 | 281 | ATRX_E25 | ATRX | | |
| x | 76855144 | 76855344 | 201 | ATRX_E24 | ATRX | | |
| x | 76855847 | 76856087 | 241 | ATRX_E23 | ATRX | | |
| x | 76872019 | 76872259 | 241 | ATRX_E22 | ATRX | | |
| x | 76874221 | 76874501 | 281 | ATRX_E21 | ATRX | | |
| x | 76875811 | 76876051 | 241 | ATRX_E20 | ATRX | | |
| x | 76888643 | 76888923 | 281 | ATRX_E19 | ATRX | | |
| x | 76889006 | 76889246 | 241 | ATRX_E18 | ATRX | | |
| x | 76890019 | 76890259 | 241 | ATRX_E17 | ATRX | | |
| x | 76891356 | 76891596 | 241 | ATRX_E16 | ATRX | | |
| x | 76907581 | 76907781 | 201 | ATRX_E15 | ATRX | | |
| x | 76907839 | 76907959 | 121 | ATRX_E15 | ATRX | | |
| x | 76909538 | 76909738 | 201 | ATRX_E14 | ATRX | | |
| x | 76911966 | 76912166 | 201 | ATRX_E13 | ATRX | | |
| x | 76918818 | 76919098 | 281 | ATRX_E12 | ATRX | | |
| x | 76920080 | 76920320 | 241 | ATRX_E11 | ATRX | | |
| x | 76931656 | 76931856 | 201 | ATRX_E10 | ATRX | | |
| x | 76936995 | 76938275 | 1281 | ATRX_E9 | ATRX | | |
| x | 76938293 | 76940093 | 1801 | ATRX_E9 | ATRX | | |
| x | 76940384 | 76940544 | 161 | ATRX_E8 | ATRX | | |
| x | 76944245 | 76944485 | 241 | ATRX_E7 | ATRX | | |
| x | 76949249 | 76949489 | 241 | ATRX_E6 | ATRX | | |
| x | 76952008 | 76952248 | 241 | ATRX_E5 | ATRX | | |
| x | 76953016 | 76953176 | 161 | ATRX_E4 | ATRX | | |
| x | 76954009 | 76954169 | 161 | ATRX_E3 | ATRX | | |
| x | 76972543 | 76972783 | 241 | ATRX_E2 | ATRX | | |
| x | 77041377 | 77041577 | 201 | ATRX_E1 | ATRX | | |
| 12 | 122459979 | 122460156 | 178 | NM_020993 | BCL7A | | |
| 12 | 122468535 | 122468708 | 174 | NM_020993 | BCL7A | | |
| 12 | 122473137 | 122473390 | 254 | NM_020993 | BCL7A | | |
| 12 | 122481738 | 122482000 | 263 | NM_020993 | BCL7A | | |
| 12 | 122492663 | 122492930 | 268 | NM_020993 | BCL7A | | |
| 12 | 122496962 | 122497195 | 234 | NM_020993 | BCL7A | | |
| 7 | 140481376 | 140481493 | 118 | NM_004333 | BRAF | | |
| 7 | 140477791 | 140477875 | 85 | NM_004333 | BRAF | | |
| 7 | 140476712 | 140476888 | 177 | NM_004333 | BRAF | | |
| 7 | 140453987 | 140454033 | 47 | NM_004333 | BRAF | | |
| 7 | 140453075 | 140453193 | 119 | NM_004333 | BRAF | | |
| 11 | 69455855 | 69456279 | 425 | NM_053056 | CCND1 | | |
| 11 | 69457799 | 69458014 | 216 | NM_053056 | CCND1 | | |
| 11 | 69458600 | 69458759 | 160 | NM_053056 | CCND1 | | |
| 11 | 69462762 | 69462910 | 149 | NM_053056 | CCND1 | | |
| 12 | 6702257 | 6702394 | 138 | CDH4 | CDH4 | | |
| 12 | 6701559 | 6701732 | 174 | CDH4 | CDH4 | | |
| 12 | 6700632 | 6700749 | 118 | CDH4 | CDH4 | | |
| 12 | 6696878 | 6697115 | 238 | CDH4 | CDH4 | | |
| 12 | 12870642 | 12871310 | 669 | CDKN1B | CDKN1B | | |
| 12 | 12871670 | 12871939 | 270 | CDKN1B | CDKN1B | | |
| 12 | 12873949 | 12874189 | 241 | CDKN1B | CDKN1B | | |
| 1 | 51436030 | 51436169 | 140 | CDKN2C | CDKN2C | | |
| 1 | 51439565 | 51440305 | 741 | CDKN2C | CDKN2C | | |
| 16 | 3777696 | 3778416 | 720 | CREBBP_E31 | CREBBP | | |
| 16 | 3778452 | 3779892 | 1440 | CREBBP_E31 | CREBBP | | |
| 16 | 3781173 | 3781493 | 320 | CREBBP_E30 | CREBBP | | |
| 16 | 3781705 | 3781865 | 160 | CREBBP_E29 | CREBBP | | |
| 16 | 3781902 | 3782102 | 200 | CREBBP_E29 | CREBBP | | |
| 16 | 3785980 | 3786260 | 280 | CREBBP_E28 | CREBBP | | |
| 16 | 3786593 | 3786873 | 280 | CREBBP_E27 | CREBBP | | |
| 16 | 3788496 | 3788736 | 240 | CREBBP_E26 | CREBBP | | |
| 16 | 3789531 | 3789771 | 240 | CREBBP_E25 | CREBBP | | |
| 16 | 3790334 | 3790614 | 280 | CREBBP_E24 | CREBBP | | |
| 16 | 3794848 | 3795008 | 160 | CREBBP_E23 | CREBBP | | |
| 16 | 3795216 | 3795416 | 200 | CREBBP_E22 | CREBBP | | |
| 16 | 3799575 | 3799735 | 160 | CREBBP_E21 | CREBBP | | |
| 16 | 3801666 | 3801866 | 200 | CREBBP_E20 | CREBBP | | |
| 16 | 3807232 | 3807432 | 200 | CREBBP_E19 | CREBBP | | |
| 16 | 3807789 | 3808069 | 280 | CREBBP_E18 | CREBBP | | |
| 16 | 3808793 | 3809033 | 240 | CREBBP_E17 | CREBBP | | |
| 16 | 3817695 | 3817935 | 240 | CREBBP_E16 | CREBBP | | |
| 16 | 3819124 | 3819404 | 280 | CREBBP_E15 | CREBBP | | |
| 16 | 3820558 | 3820998 | 440 | CREBBP_E14 | CREBBP | | |
| 16 | 3823701 | 3823981 | 280 | CREBBP_E13 | CREBBP | | |
| 16 | 3824511 | 3824751 | 240 | CREBBP_E12 | CREBBP | | |
| 16 | 3827527 | 3827687 | 160 | CREBBP_E11 | CREBBP | | |
| 16 | 3827957 | 3828237 | 280 | CREBBP_E10 | CREBBP | | |
| 16 | 3828639 | 3828879 | 240 | CREBBP_E9 | CREBBP | | |
| 16 | 3830685 | 3830925 | 240 | CREBBP_E8 | CREBBP | | |
| 16 | 3831155 | 3831355 | 200 | CREBBP_E7 | CREBBP | | |
| 16 | 3832665 | 3832945 | 280 | CREBBP_E6 | CREBBP | | |
| 16 | 3841918 | 3842158 | 240 | CREBBP_E5 | CREBBP | | |
| 16 | 3843366 | 3843646 | 280 | CREBBP_E4 | CREBBP | | |
| 16 | 3860551 | 3860831 | 280 | CREBBP_E3 | CREBBP | | |
| 16 | 3900273 | 3901033 | 760 | CREBBP_E2 | CREBBP | | |
| 16 | 3929814 | 3929934 | 120 | CREBBP_E1 | CREBBP | | |
| 2 | 136872283 | 136873684 | 1402 | CXCR4 | CXCR4 | | |
| 2 | 136875496 | 136875761 | 266 | CXCR4 | CXCR4 | | |
| 16 | 50776193 | 50776414 | 222 | NM_015247 | CYLD | | |
| 16 | 50783564 | 50784133 | 570 | NM_015247 | CYLD | | |
| 16 | 50785482 | 50785819 | 338 | NM_015247 | CYLD | | |
| 16 | 50788227 | 50788410 | 184 | NM_015247 | CYLD | | |
| 16 | 50809011 | 50809183 | 173 | NM_015247 | CYLD | | |
| 16 | 50810052 | 50810217 | 166 | NM_015247 | CYLD | | |
| 16 | 50811720 | 50811910 | 191 | NM_015247 | CYLD | | |
| 16 | 50813536 | 50813989 | 454 | NM_015247 | CYLD | | |
| 16 | 50815124 | 50815351 | 228 | NM_015247 | CYLD | | |
| 16 | 50816186 | 50816436 | 251 | NM_015247 | CYLD | | |
| 16 | 50818204 | 50818432 | 229 | NM_015247 | CYLD | | |
| 16 | 50820719 | 50820939 | 221 | NM_015247 | CYLD | | |
| 16 | 50821652 | 50821857 | 206 | NM_015247 | CYLD | | |
| 16 | 50825451 | 50825650 | 200 | NM_015247 | CYLD | | |
| 16 | 50826426 | 50826724 | 299 | NM_015247 | CYLD | | |
| 16 | 50827397 | 50827602 | 206 | NM_015247 | CYLD | | |
| 16 | 50828066 | 50828458 | 393 | NM_015247 | CYLD | | |
| 16 | 50829330 | 50829528 | 199 | NM_015247 | CYLD | | |
| 16 | 50830188 | 50830492 | 305 | NM_015247 | CYLD | | |
| 13 | 73336061 | 73336275 | 215 | NM_014953 | DIS3 | | |
| 13 | 73337589 | 73337745 | 157 | NM_014953 | DIS3 | | |
| 13 | 73340110 | 73340196 | 87 | NM_014953 | DIS3 | | |
| 13 | 73342923 | 73343050 | 128 | NM_014953 | DIS3 | | |
| 13 | 73345042 | 73345126 | 85 | NM_014953 | DIS3 | | |
| 13 | 73345219 | 73345283 | 65 | NM_014953 | DIS3 | | |
| 13 | 73345933 | 73346034 | 102 | NM_014953 | DIS3 | | |
| 13 | 73346297 | 73346413 | 117 | NM_014953 | DIS3 | | |
| 13 | 73346831 | 73346977 | 147 | NM_014953 | DIS3 | | |
| 13 | 73347822 | 73347959 | 138 | NM_014953 | DIS3 | | |
| 13 | 73348084 | 73348197 | 114 | NM_014953 | DIS3 | | |
| 13 | 73349349 | 73349513 | 165 | NM_014953 | DIS3 | | |
| 13 | 73350063 | 73350230 | 168 | NM_014953 | DIS3 | | |
| 13 | 73351558 | 73351631 | 74 | NM_014953 | DIS3 | | |
| 13 | 73352325 | 73352518 | 194 | NM_014953 | DIS3 | | |
| 13 | 73354984 | 73355141 | 158 | NM_014953 | DIS3 | | |
| 13 | 73355743 | 73356071 | 329 | NM_014953 | DIS3 | | |
| 2 | 25457098 | 25457338 | 241 | DNMT3A_E23 | DNMT3A | | |
| 2 | 25457098 | 25457338 | 241 | DNMT3A_E23 | DNMT3A | | |
| 2 | 25458514 | 25458754 | 241 | DNMT3A_E22 | DNMT3A | | |
| 2 | 25458514 | 25458754 | 241 | DNMT3A_E22 | DNMT3A | | |
| 2 | 25459739 | 25459939 | 201 | DNMT3A_E21 | DNMT3A | | |
| 2 | 25459739 | 25459939 | 201 | DNMT3A_E21 | DNMT3A | | |
| 2 | 25461941 | 25462141 | 201 | DNMT3A_E20 | DNMT3A | | |
| 2 | 25461941 | 25462141 | 201 | DNMT3A_E20 | DNMT3A | | |
| 2 | 25463104 | 25463384 | 281 | DNMT3A_E19 | DNMT3A | | |
| 2 | 25463104 | 25463384 | 281 | DNMT3A_E19 | DNMT3A | | |
| 2 | 25463453 | 25463653 | 201 | DNMT3A_E18 | DNMT3A | | |
| 2 | 25463453 | 25463653 | 201 | DNMT3A_E18 | DNMT3A | | |
| 2 | 25464383 | 25464623 | 241 | DNMT3A_E17 | DNMT3A | | |
| 2 | 25464383 | 25464623 | 241 | DNMT3A_E17 | DNMT3A | | |
| 2 | 25466708 | 25466908 | 201 | DNMT3A_E16 | DNMT3A | | |
| 2 | 25466708 | 25466908 | 201 | DNMT3A_E16 | DNMT3A | | |
| 2 | 25466975 | 25467255 | 281 | DNMT3A_E15 | DNMT3A | | |
| 2 | 25466975 | 25467255 | 281 | DNMT3A_E15 | DNMT3A | | |
| 2 | 25467344 | 25467584 | 241 | DNMT3A_E14 | DNMT3A | | |
| 2 | 25467344 | 25467584 | 241 | DNMT3A_E14 | DNMT3A | | |
| 2 | 25468061 | 25468261 | 201 | DNMT3A_E13 | DNMT3A | | |
| 2 | 25468061 | 25468261 | 201 | DNMT3A_E13 | DNMT3A | | |
| 2 | 25468830 | 25469243 | 414 | DNMT3A_E11 | DNMT3A | | |
| 2 | 25468830 | 25469243 | 414 | DNMT3A_E11 | DNMT3A | | |
| 2 | 25469426 | 25469706 | 281 | DNMT3A_E10 | DNMT3A | | |
| 2 | 25469426 | 25469706 | 281 | DNMT3A_E10 | DNMT3A | | |
| 2 | 25469873 | 25470073 | 201 | DNMT3A_E9 | DNMT3A | | |
| 2 | 25469873 | 25470073 | 201 | DNMT3A_E9 | DNMT3A | | |
| 2 | 25470398 | 25470678 | 281 | DNMT3A_E8 | DNMT3A | | |
| 2 | 25470398 | 25470678 | 281 | DNMT3A_E8 | DNMT3A | | |
| 2 | 25470893 | 25471133 | 241 | DNMT3A_E7 | DNMT3A | | |
| 2 | 25470893 | 25471133 | 241 | DNMT3A_E7 | DNMT3A | | |
| 2 | 25497762 | 25498002 | 241 | DNMT3A_E6 | DNMT3A | | |
| 2 | 25497762 | 25498002 | 241 | DNMT3A_E6 | DNMT3A | | |
| 2 | 25498310 | 25498470 | 161 | DNMT3A_E5 | DNMT3A | | |
| 2 | 25498310 | 25498470 | 161 | DNMT3A_E5 | DNMT3A | | |
| 2 | 25504304 | 25505024 | 721 | DNMT3A_E4 | DNMT3A | | |
| 2 | 25504304 | 25505024 | 721 | DNMT3A_E4 | DNMT3A | | |
| 2 | 25505113 | 25505593 | 481 | DNMT3A_E4 | DNMT3A | | |
| 2 | 25505113 | 25505593 | 481 | DNMT3A_E4 | DNMT3A | | |
| 2 | 25522959 | 25523159 | 201 | DNMT3A_E3 | DNMT3A | | |
| 2 | 25522959 | 25523159 | 201 | DNMT3A_E3 | DNMT3A | | |
| 2 | 25536717 | 25536917 | 201 | DNMT3A_E2 | DNMT3A | | |
| 2 | 25536717 | 25536917 | 201 | DNMT3A_E2 | DNMT3A | | |
| 5 | 137801403 | 137801638 | 236 | NM_001964 | EGR1 | | |
| 5 | 137801680 | 137801801 | 122 | NM_001964 | EGR1 | | |
| 5 | 137802418 | 137803822 | 1405 | NM_001964 | EGR1 | | |
| 5 | 137803943 | 137804146 | 204 | NM_001964 | EGR1 | | |
| 22 | 41488955 | 41489155 | 201 | EP300_E1 | EP300 | | |
| 22 | 41513167 | 41513847 | 681 | EP300_E2 | EP300 | | |
| 22 | 41521815 | 41522095 | 281 | EP300_E3 | EP300 | | |
| 22 | 41523481 | 41523761 | 281 | EP300_E4 | EP300 | | |
| 22 | 41525830 | 41526070 | 241 | EP300_E5 | EP300 | | |
| 22 | 41527374 | 41527654 | 281 | EP300_E6 | EP300 | | |
| 22 | 41531763 | 41531963 | 201 | EP300_E7 | EP300 | | |
| 22 | 41533605 | 41533845 | 241 | EP300_E8 | EP300 | | |
| 22 | 41536082 | 41536322 | 241 | EP300_E9 | EP300 | | |
| 22 | 41536998 | 41537278 | 281 | EP300_E10 | EP300 | | |
| 22 | 41542649 | 41542849 | 201 | EP300_E11 | EP300 | | |
| 22 | 41543775 | 41544015 | 241 | EP300_E12 | EP300 | | |
| 22 | 41544990 | 41545230 | 241 | EP300_E13 | EP300 | | |
| 22 | 41545743 | 41546223 | 481 | EP300_E14 | EP300 | | |
| 22 | 41547786 | 41548066 | 281 | EP300_E15 | EP300 | | |
| 22 | 41548161 | 41548401 | 241 | EP300_E16 | EP300 | | |
| 22 | 41550937 | 41551177 | 241 | EP300_E17 | EP300 | | |
| 22 | 41553152 | 41553432 | 281 | EP300_E18 | EP300 | | |
| 22 | 41554359 | 41554559 | 201 | EP300_E19 | EP300 | | |
| 22 | 41556585 | 41556785 | 201 | EP300_E20 | EP300 | | |
| 22 | 41558674 | 41558834 | 161 | EP300_E21 | EP300 | | |
| 22 | 41559995 | 41560195 | 201 | EP300_E22 | EP300 | | |
| 22 | 41562556 | 41562716 | 161 | EP300_E23 | EP300 | | |
| 22 | 41564387 | 41564667 | 281 | EP300_E24 | EP300 | | |
| 22 | 41564677 | 41564917 | 241 | EP300_E25 | EP300 | | |
| 22 | 41565443 | 41565683 | 241 | EP300_E26 | EP300 | | |
| 22 | 41566352 | 41566632 | 281 | EP300_E27 | EP300 | | |
| 22 | 41568444 | 41568724 | 281 | EP300_E28 | EP300 | | |
| 22 | 41569567 | 41569847 | 281 | EP300_E29 | EP300 | | |
| 22 | 41572231 | 41572551 | 321 | EP300_E30 | EP300 | | |
| 22 | 41572768 | 41574968 | 2201 | EP300_E31 | EP300 | | |
| 17 | 41622926 | 41623036 | 111 | ETV4 | ETV4 | | |
| 17 | 41622642 | 41622735 | 94 | ETV4 | ETV4 | | |
| 17 | 41622343 | 41622390 | 48 | ETV4 | ETV4 | | |
| 17 | 41613794 | 41613847 | 54 | ETV4 | ETV4 | | |
| 17 | 41611227 | 41611353 | 127 | ETV4 | ETV4 | | |
| 17 | 41610555 | 41610716 | 162 | ETV4 | ETV4 | | |
| 17 | 41610042 | 41610307 | 266 | ETV4 | ETV4 | | |
| 17 | 41607475 | 41607549 | 75 | ETV4 | ETV4 | | |
| 17 | 41607252 | 41607320 | 69 | ETV4 | ETV4 | | |
| 17 | 41606872 | 41607044 | 173 | ETV4 | ETV4 | | |
| 17 | 41606503 | 41606604 | 102 | ETV4 | ETV4 | | |
| 17 | 41605212 | 41606111 | 900 | ETV4 | ETV4 | | |
| 1 | 118165412 | 118166711 | 1300 | NM_017709 | FAM46C | | |
| 4 | 1803347 | 1803470 | 124 | FGFR3 | FGFR3 | | |
| 4 | 1803562 | 1803752 | 191 | FGFR3 | FGFR3 | | |
| 4 | 1804641 | 1804791 | 151 | FGFR3 | FGFR3 | | |
| 4 | 1807778 | 1807900 | 123 | FGFR3 | FGFR3 | | |
| 1 | 78432733 | 78432785 | 53 | FUBP1 | FUBP1 | | |
| 1 | 78432568 | 78432639 | 72 | FUBP1 | FUBP1 | | |
| 1 | 78432378 | 78432435 | 58 | FUBP1 | FUBP1 | | |
| 1 | 78430753 | 78430915 | 163 | FUBP1 | FUBP1 | | |
| 1 | 78429259 | 78429400 | 142 | FUBP1 | FUBP1 | | |
| 1 | 78428455 | 78428615 | 161 | FUBP1 | FUBP1 | | |
| 1 | 78425869 | 78425948 | 80 | FUBP1 | FUBP1 | | |
| 1 | 78422257 | 78422385 | 129 | FUBP1 | FUBP1 | | |
| 1 | 78420940 | 78421014 | 75 | FUBP1 | FUBP1 | | |
| 1 | 78414840 | 78414985 | 146 | FUBP1 | FUBP1 | | |
| 6 | 26055968 | 26056699 | 732 | HIST1H1C | HIST1H1C | | |
| 6 | 26156586 | 26157317 | 732 | NM_005321 | HIST1H1E | | |
| 6 | 26271146 | 26271612 | 467 | HIST1H3G | HIST1H3G | | |
| 6 | 27777842 | 27778314 | 473 | HIST1H3H | HIST1H3H | | |
| 2 | 209101748 | 209101972 | 225 | NM_005896 | IDH1 | | |
| 2 | 209103672 | 209103998 | 327 | NM_005896 | IDH1 | | |
| 2 | 209104479 | 209104759 | 281 | NM_005896 | IDH1 | | |
| 2 | 209106673 | 209106955 | 283 | NM_005896 | IDH1 | | |
| 2 | 209108010 | 209108401 | 392 | NM_005896 | IDH1 | | |
| 2 | 209109989 | 209110233 | 245 | NM_005896 | IDH1 | | |
| 2 | 209112997 | 209113442 | 446 | NM_005896 | IDH1 | | |
| 2 | 209116076 | 209116365 | 290 | NM_005896 | IDH1 | | |
| 15 | 90627326 | 90627446 | 121 | NM_002168 | IDH2 | | |
| 15 | 90627497 | 90627665 | 169 | NM_002168 | IDH2 | | |
| 15 | 90627993 | 90628724 | 732 | NM_002168 | IDH2 | | |
| 15 | 90630266 | 90630552 | 287 | NM_002168 | IDH2 | | |
| 15 | 90630615 | 90630829 | 215 | NM_002168 | IDH2 | | |
| 15 | 90631471 | 90632002 | 532 | NM_002168 | IDH2 | | |
| 15 | 90633691 | 90633954 | 264 | NM_002168 | IDH2 | | |
| 15 | 90634718 | 90634896 | 179 | NM_002168 | IDH2 | | |
| 15 | 90645453 | 90645690 | 238 | NM_002168 | IDH2 | | |
| 15 | 99439986 | 99440134 | 149 | IGF1R | IGF1R | | |
| 15 | 99465377 | 99465660 | 284 | IGF1R | IGF1R | | |
| 15 | 99478053 | 99478282 | 230 | IGF1R | IGF1R | | |
| 6 | 391710 | 391959 | 250 | NM_002460 | IRF4 | | |
| 6 | 393061 | 393385 | 325 | NM_002460 | IRF4 | | |
| 6 | 394751 | 395093 | 343 | NM_002460 | IRF4 | | |
| 6 | 395754 | 395995 | 242 | NM_002460 | IRF4 | | |
| 6 | 397093 | 397365 | 273 | NM_002460 | IRF4 | | |
| 6 | 398733 | 398989 | 257 | NM_002460 | IRF4 | | |
| 6 | 401399 | 401842 | 444 | NM_002460 | IRF4 | | |
| 6 | 404972 | 405216 | 245 | NM_002460 | IRF4 | | |
| 6 | 406633 | 406753 | 121 | NM_002460 | IRF4 | | |
| 6 | 406755 | 406875 | 121 | NM_002460 | IRF4 | | |
| 6 | 407451 | 407633 | 183 | NM_002460 | IRF4 | | |
| x | 53230732 | 53230926 | 195 | KDM5C | KDM5C | | |
| x | 53223321 | 53223920 | 600 | KDM5C | KDM5C | | |
| x | 44918492 | 44918711 | 220 | KDM6A | KDM6A | | |
| X | 44922667 | 44923062 | 396 | KDM6A | KDM6A | | |
| x | 44966655 | 44966781 | 127 | KDM6A | KDM6A | | |
| 19 | 36220060 | 36220197 | 138 | KMT2B | KMT2B | | |
| 19 | 36220868 | 36221026 | 159 | KMT2B | KMT2B | | |
| 19 | 36221243 | 36221363 | 121 | KMT2B | KMT2B | | |
| 19 | 36221439 | 36221517 | 79 | KMT2B | KMT2B | | |
| 19 | 36221608 | 36221768 | 161 | KMT2B | KMT2B | | |
| 19 | 36222809 | 36223036 | 228 | KMT2B | KMT2B | | |
| 19 | 36223116 | 36224409 | 1294 | KMT2B | KMT2B | | |
| 7 | 151833800 | 151834012 | 213 | NM_170606 | KMT2C | | |
| 7 | 151835866 | 151836044 | 179 | NM_170606 | KMT2C | | |
| 7 | 151836261 | 151836443 | 183 | NM_170606 | KMT2C | | |
| 7 | 151836732 | 151836908 | 177 | NM_170606 | KMT2C | | |
| 7 | 151841763 | 151842017 | 255 | NM_170606 | KMT2C | | |
| 7 | 151842207 | 151842440 | 234 | NM_170606 | KMT2C | | |
| 7 | 151843627 | 151843840 | 214 | NM_170606 | KMT2C | | |
| 7 | 151845107 | 151846264 | 1158 | NM_170606 | KMT2C | | |
| 7 | 151847892 | 151848119 | 228 | NM_170606 | KMT2C | | |
| 7 | 151848507 | 151848680 | 174 | NM_170606 | KMT2C | | |
| 7 | 151849751 | 151850071 | 321 | NM_170606 | KMT2C | | |
| 7 | 151851084 | 151851267 | 184 | NM_170606 | KMT2C | | |
| 7 | 151851270 | 151851545 | 276 | NM_170606 | KMT2C | | |
| 7 | 151852958 | 151853188 | 231 | NM_170606 | KMT2C | | |
| 7 | 151853220 | 151853459 | 240 | NM_170606 | KMT2C | | |
| 7 | 151854831 | 151855016 | 186 | NM_170606 | KMT2C | | |
| 7 | 151855885 | 151856189 | 305 | NM_170606 | KMT2C | | |
| 7 | 151859164 | 151860943 | 1780 | NM_170606 | KMT2C | | |
| 7 | 151864209 | 151864473 | 265 | NM_170606 | KMT2C | | |
| 7 | 151866148 | 151866345 | 198 | NM_170606 | KMT2C | | |
| 7 | 151868245 | 151868479 | 235 | NM_170606 | KMT2C | | |
| 7 | 151871144 | 151871384 | 241 | NM_170606 | KMT2C | | |
| 7 | 151873235 | 151875119 | 1885 | NM_170606 | KMT2C | | |
| 7 | 151876904 | 151877228 | 325 | NM_170606 | KMT2C | | |
| 7 | 151877785 | 151879689 | 1905 | NM_170606 | KMT2C | | |
| 7 | 151880051 | 151880300 | 250 | NM_170606 | KMT2C | | |
| 7 | 151882535 | 151882746 | 212 | NM_170606 | KMT2C | | |
| 7 | 151884331 | 151884620 | 290 | NM_170606 | KMT2C | | |
| 7 | 151884763 | 151884953 | 191 | NM_170606 | KMT2C | | |
| 7 | 151891054 | 151891392 | 339 | NM_170606 | KMT2C | | |
| 7 | 151891506 | 151891671 | 166 | NM_170606 | KMT2C | | |
| 7 | 151892894 | 151893101 | 208 | NM_170606 | KMT2C | | |
| 7 | 151896361 | 151896576 | 216 | NM_170606 | KMT2C | | |
| 7 | 151899964 | 151900214 | 251 | NM_170606 | KMT2C | | |
| 7 | 151902110 | 151902332 | 223 | NM_170606 | KMT2C | | |
| 7 | 151904382 | 151904518 | 137 | NM_170606 | KMT2C | | |
| 7 | 151917593 | 151917833 | 241 | NM_170606 | KMT2C | | |
| 7 | 151919042 | 151919210 | 169 | NM_170606 | KMT2C | | |
| 7 | 151919652 | 151919772 | 121 | NM_170606 | KMT2C | | |
| 7 | 151921061 | 151921301 | 241 | NM_170606 | KMT2C | | |
| 7 | 151921490 | 151921730 | 241 | NM_170606 | KMT2C | | |
| 7 | 151926999 | 151927119 | 121 | NM_170606 | KMT2C | | |
| 7 | 151927295 | 151927415 | 121 | NM_170606 | KMT2C | | |
| 7 | 151932899 | 151933019 | 121 | NM_170606 | KMT2C | | |
| 7 | 151933144 | 151933235 | 92 | NM_170606 | KMT2C | | |
| 7 | 151935791 | 151935911 | 121 | NM_170606 | KMT2C | | |
| 7 | 151944977 | 151945757 | 781 | NM_170606 | KMT2C | | |
| 7 | 151946951 | 151947121 | 171 | NM_170606 | KMT2C | | |
| 7 | 151947890 | 151948056 | 167 | NM_170606 | KMT2C | | |
| 7 | 151949016 | 151949184 | 169 | NM_170606 | KMT2C | | |
| 7 | 151949581 | 151949817 | 237 | NM_170606 | KMT2C | | |
| 7 | 151960005 | 151960235 | 231 | NM_170606 | KMT2C | | |
| 7 | 151962111 | 151962299 | 189 | NM_170606 | KMT2C | | |
| 7 | 151970734 | 151971003 | 270 | NM_170606 | KMT2C | | |
| 7 | 152007021 | 152007203 | 183 | NM_170606 | KMT2C | | |
| 7 | 152008850 | 152009038 | 189 | NM_170606 | KMT2C | | |
| 7 | 152012164 | 152012428 | 265 | NM_170606 | KMT2C | | |
| 7 | 152027651 | 152027837 | 187 | NM_170606 | KMT2C | | |
| 7 | 152041261 | 152041382 | 122 | NM_170606 | KMT2C | | |
| 7 | 152055605 | 152055779 | 175 | NM_170606 | KMT2C | | |
| 7 | 152132706 | 152132909 | 204 | NM_170606 | KMT2C | | |
| 12 | 49433507 | 49435318 | 1812 | KMT2D | KMT2D | | |
| 12 | 49433218 | 49433400 | 183 | KMT2D | KMT2D | | |
| 12 | 49433005 | 49433141 | 137 | KMT2D | KMT2D | | |
| 12 | 49430908 | 49432772 | 1865 | KMT2D | KMT2D | | |
| 12 | 49428595 | 49428718 | 124 | KMT2D | KMT2D | | |
| 12 | 49428365 | 49428449 | 85 | KMT2D | KMT2D | | |
| 12 | 49428193 | 49428259 | 67 | KMT2D | KMT2D | | |
| 12 | 49427850 | 49428082 | 233 | KMT2D | KMT2D | | |
| 12 | 49424958 | 49427747 | 2790 | KMT2D | KMT2D | | |
| 12 | 49424676 | 49424816 | 141 | KMT2D | KMT2D | | |
| 12 | 49424384 | 49424551 | 168 | KMT2D | KMT2D | | |
| 12 | 49424063 | 49424222 | 160 | KMT2D | KMT2D | | |
| 12 | 49423184 | 49423259 | 76 | KMT2D | KMT2D | | |
| 12 | 49422844 | 49423019 | 176 | KMT2D | KMT2D | | |
| 12 | 49422611 | 49422741 | 131 | KMT2D | KMT2D | | |
| 12 | 49421792 | 49421924 | 133 | KMT2D | KMT2D | | |
| 12 | 49421586 | 49421713 | 128 | KMT2D | KMT2D | | |
| 12 | 49419965 | 49421105 | 1141 | KMT2D | KMT2D | | |
| 12 | 49418593 | 49418729 | 137 | KMT2D | KMT2D | | |
| 12 | 49418361 | 49418491 | 131 | KMT2D | KMT2D | | |
| 12 | 49416373 | 49416658 | 286 | KMT2D | KMT2D | | |
| 12 | 49416063 | 49416136 | 74 | KMT2D | KMT2D | | |
| 12 | 49415826 | 49415934 | 109 | KMT2D | KMT2D | | |
| 12 | 25398208 | 25398329 | 122 | NM_033360 | KRAS | | |
| 12 | 25380168 | 25380346 | 179 | NM_033360 | KRAS | | |
| 12 | 25378548 | 25378707 | 160 | NM_033360 | KRAS | | |
| 6 | 31548447 | 31548988 | 542 | NM_009588 | LTB | | |
| 6 | 31549222 | 31549416 | 195 | NM_009588 | LTB | | |
| 6 | 31549566 | 31549770 | 205 | NM_009588 | LTB | | |
| 6 | 31549965 | 31550234 | 270 | NM_009588 | LTB | | |
| 16 | 79633799 | 79634920 | 1122 | MAF (translated) | MAF | | |
| 20 | 39316519 | 39317490 | 972 | MAFB (translated) | MAFB | | |
| 14 | 65569022 | 65569188 | 167 | NM_197957 | MAX | | |
| 14 | 65560426 | 65560533 | 108 | NM_197957 | MAX | | |
| 14 | 65544631 | 65544754 | 124 | NM_197957 | MAX | | |
| 14 | 65541330 | 65543381 | 2052 | NM_197957 | MAX | | |
| 11 | 118370018 | 118370135 | 118 | KMT2A | MLL | | |
| 11 | 118370550 | 118370628 | 79 | KMT2A | MLL | | |
| 11 | 118371702 | 118371862 | 161 | KMT2A | MLL | | |
| 11 | 118372387 | 118372572 | 186 | KMT2A | MLL | | |
| 11 | 118373113 | 118377361 | 4249 | KMT2A | MLL | | |
| 3 | 38181860 | 38182100 | 241 | MYD88_E3 | MYD88 | | |
| 3 | 38182193 | 38182393 | 201 | MYD88_E4 | MYD88 | | |
| 3 | 38182559 | 38182839 | 281 | MYD88_E5 | MYD88 | | |
| 17 | 16004564 | 16005071 | 508 | NCOR1 | NCOR1 | | |
| 17 | 15968799 | 15969008 | 210 | NCOR1 | NCOR1 | | |
| 17 | 29422297 | 29422457 | 161 | NF1_E1 | NF1 | | |
| 17 | 29482952 | 29483192 | 241 | NF1_E2 | NF1 | | |
| 17 | 29485969 | 29486169 | 201 | NF1_E3 | NF1 | | |
| 17 | 29490178 | 29490418 | 241 | NF1_E4 | NF1 | | |
| 17 | 29496861 | 29497061 | 201 | NF1_E5 | NF1 | | |
| 17 | 29508393 | 29508553 | 161 | NF1_E6 | NF1 | | |
| 17 | 29508665 | 29508865 | 201 | NF1_E7 | NF1 | | |
| 17 | 29509464 | 29509744 | 281 | NF1_E8 | NF1 | | |
| 17 | 29527386 | 29527666 | 281 | NF1_E9 | NF1 | | |
| 17 | 29527995 | 29528235 | 241 | NF1_E10 | NF1 | | |
| 17 | 29528365 | 29528565 | 201 | NF1_E11 | NF1 | | |
| 17 | 29533203 | 29533443 | 241 | NF1_E12 | NF1 | | |
| 17 | 29541415 | 29541655 | 241 | NF1_E13 | NF1 | | |
| 17 | 29545959 | 29546199 | 241 | NF1_E14 | NF1 | | |
| 17 | 29548776 | 29548976 | 201 | NF1_E15 | NF1 | | |
| 17 | 29550403 | 29550643 | 241 | NF1_E16 | NF1 | | |
| 17 | 29552050 | 29552330 | 281 | NF1_E17 | NF1 | | |
| 17 | 29553437 | 29553717 | 281 | NF1_E18 | NF1 | | |
| 17 | 29554172 | 29554372 | 201 | NF1_E19 | NF1 | | |
| 17 | 29554482 | 29554682 | 201 | NF1_E20 | NF1 | | |
| 17 | 29556022 | 29556502 | 481 | NF1_E21 | NF1 | | |
| 17 | 29556802 | 29557042 | 241 | NF1_E22 | NF1 | | |
| 17 | 29557218 | 29557458 | 241 | NF1_E23 | NF1 | | |
| 17 | 29557801 | 29558001 | 201 | NF1_E24 | NF1 | | |
| 17 | 29559068 | 29559308 | 241 | NF1_E25 | NF1 | | |
| 17 | 29559668 | 29559948 | 281 | NF1_E26 | NF1 | | |
| 17 | 29560005 | 29560245 | 241 | NF1_E27 | NF1 | | |
| 17 | 29562569 | 29562849 | 281 | NF1_E28 | NF1 | | |
| 17 | 29562887 | 29563087 | 201 | NF1_E29 | NF1 | | |
| 17 | 29575949 | 29576189 | 241 | NF1_E30 | NF1 | | |
| 17 | 29579906 | 29580066 | 161 | NF1_E31 | NF1 | | |
| 17 | 29585300 | 29585580 | 281 | NF1_E32 | NF1 | | |
| 17 | 29585998 | 29586198 | 201 | NF1_E33 | NF1 | | |
| 17 | 29587339 | 29587579 | 241 | NF1_E34 | NF1 | | |
| 17 | 29588681 | 29588921 | 241 | NF1_E35 | NF1 | | |
| 17 | 29592181 | 29592421 | 241 | NF1_E36 | NF1 | | |
| 17 | 29652813 | 29653293 | 481 | NF1_E37 | NF1 | | |
| 17 | 29654506 | 29654866 | 361 | NF1_E38 | NF1 | | |
| 17 | 29657294 | 29657534 | 241 | NF1_E39 | NF1 | | |
| 17 | 29661832 | 29662072 | 241 | NF1_E40 | NF1 | | |
| 17 | 29663300 | 29663540 | 241 | NF1_E41 | NF1 | | |
| 17 | 29663632 | 29663952 | 321 | NF1_E42 | NF1 | | |
| 17 | 29664372 | 29664612 | 241 | NF1_E43 | NF1 | | |
| 17 | 29664787 | 29664947 | 161 | NF1_E44 | NF1 | | |
| 17 | 29664982 | 29665222 | 241 | NF1_E45 | NF1 | | |
| 17 | 29665672 | 29665872 | 201 | NF1_E46 | NF1 | | |
| 17 | 29667472 | 29667712 | 241 | NF1_E47 | NF1 | | |
| 17 | 29669969 | 29670209 | 241 | NF1_E48 | NF1 | | |
| 17 | 29676116 | 29676356 | 241 | NF1_E49 | NF1 | | |
| 17 | 29677148 | 29677388 | 241 | NF1_E50 | NF1 | | |
| 17 | 29679194 | 29679474 | 281 | NF1_E51 | NF1 | | |
| 17 | 29683418 | 29683658 | 241 | NF1_E52 | NF1 | | |
| 17 | 29683922 | 29684162 | 241 | NF1_E53 | NF1 | | |
| 17 | 29684236 | 29684436 | 201 | NF1_E54 | NF1 | | |
| 17 | 29685448 | 29685688 | 241 | NF1_E55 | NF1 | | |
| 17 | 29685929 | 29686089 | 161 | NF1_E56 | NF1 | | |
| 17 | 29687492 | 29687732 | 241 | NF1_E57 | NF1 | | |
| 17 | 29700981 | 29701221 | 241 | NF1_E58 | NF1 | | |
| 14 | 35871019 | 35871400 | 382 | NM_020529 | NFKBIA | | |
| 14 | 35871558 | 35872096 | 539 | NM_020529 | NFKBIA | | |
| 14 | 35872351 | 35872585 | 235 | NM_020529 | NFKBIA | | |
| 14 | 35872790 | 35873033 | 244 | NM_020529 | NFKBIA | | |
| 14 | 35873610 | 35873862 | 253 | NM_020529 | NFKBIA | | |
| 9 | 139392010 | 139393498 | 1489 | NM_017617 | NOTCH1 | | |
| 1 | 115251077 | 115251321 | 245 | NM_002524 | NRAS | | |
| 1 | 115252126 | 115252365 | 240 | NM_002524 | NRAS | | |
| 1 | 115256373 | 115256617 | 245 | NM_002524 | NRAS | | |
| 1 | 115258578 | 115258801 | 224 | NM_002524 | NRAS | | |
| 6 | 30655824 | 30656719 | 896 | NRM | NRM | | |
| 6 | 30657053 | 30657229 | 177 | NRM | NRM | | |
| 6 | 30657824 | 30658020 | 197 | NRM | NRM | | |
| 6 | 30658619 | 30659058 | 440 | NRM | NRM | | |
| 3 | 178916547 | 178917003 | 457 | NM_006218 | PIK3CA | | |
| 3 | 178917385 | 178917744 | 360 | NM_006218 | PIK3CA | | |
| 3 | 178919010 | 178919360 | 351 | NM_006218 | PIK3CA | | |
| 3 | 178921289 | 178921601 | 313 | NM_006218 | PIK3CA | | |
| 3 | 178922264 | 178922432 | 169 | NM_006218 | PIK3CA | | |
| 3 | 178927370 | 178927547 | 178 | NM_006218 | PIK3CA | | |
| 3 | 178927878 | 178928388 | 511 | NM_006218 | PIK3CA | | |
| 3 | 178935939 | 178936157 | 219 | NM_006218 | PIK3CA | | |
| 3 | 178936897 | 178937084 | 188 | NM_006218 | PIK3CA | | |
| 3 | 178937343 | 178937543 | 201 | NM_006218 | PIK3CA | | |
| 3 | 178937688 | 178938006 | 319 | NM_006218 | PIK3CA | | |
| 3 | 178938722 | 178938957 | 236 | NM_006218 | PIK3CA | | |
| 3 | 178941837 | 178942004 | 168 | NM_006218 | PIK3CA | | |
| 3 | 178942445 | 178942658 | 214 | NM_006218 | PIK3CA | | |
| 3 | 178943652 | 178943873 | 222 | NM_006218 | PIK3CA | | |
| 3 | 178947043 | 178947244 | 202 | NM_006218 | PIK3CA | | |
| 3 | 178947713 | 178947976 | 264 | NM_006218 | PIK3CA | | |
| 3 | 178947979 | 178948177 | 199 | NM_006218 | PIK3CA | | |
| 3 | 178951812 | 178952199 | 388 | NM_006218 | PIK3CA | | |
| 3 | 178954323 | 178954505 | 183 | ENST00000263967 | PIK3CA | | |
| 17 | 58740334 | 58740934 | 601 | PPM1D_E6 | PPM1D | | |
| 1 | 12918840 | 12919151 | 312 | PRAMEF2 | PRAMEF2 | | |
| 1 | 12919548 | 12920126 | 579 | PRAMEF2 | PRAMEF2 | | |
| 6 | 106534376 | 106534552 | 177 | NM_001198 | PRDM1 | | |
| 6 | 106536028 | 106536403 | 376 | NM_001198 | PRDM1 | | |
| 6 | 106543431 | 106543703 | 273 | NM_001198 | PRDM1 | | |
| 6 | 106546444 | 106546652 | 209 | NM_001198 | PRDM1 | | |
| 6 | 106546802 | 106546922 | 121 | NM_182907 | PRDM1 | | |
| 6 | 106546927 | 106547047 | 121 | NM_182907 | PRDM1 | | |
| 6 | 106547133 | 106547440 | 308 | NM_182907 | PRDM1 | | |
| 6 | 106552642 | 106553857 | 1216 | NM_182907 | PRDM1 | | |
| 6 | 106554187 | 106554471 | 285 | NM_182907 | PRDM1 | | |
| 6 | 106554728 | 106555408 | 681 | NM_182907 | PRDM1 | | |
| 19 | 47194992 | 47195064 | 73 | PRKD2 | PRKD2 | | |
| 19 | 47193857 | 47193963 | 107 | PRKD2 | PRKD2 | | |
| 19 | 47181653 | 47181920 | 268 | PRKD2 | PRKD2 | | |
| 9 | 79319677 | 79326274 | 6598 | PRUNE2 | PRUNE2 | | |
| 9 | 79328479 | 79328637 | 159 | PRUNE2 | PRUNE2 | | |
| 9 | 79267399 | 79267599 | 201 | PRUNE2 | PRUNE2 | | |
| 9 | 79253102 | 79253204 | 103 | PRUNE2 | PRUNE2 | | |
| 12 | 112888122 | 112888316 | 195 | NM_080601 | PTPN11 | | |
| 12 | 112926828 | 112926979 | 152 | NM_080601 | PTPN11 | | |
| 3 | 141230923 | 141231178 | 255 | NM_006506 | RASA2 | | |
| 3 | 141289695 | 141289954 | 259 | NM_006506 | RASA2 | | |
| 3 | 141327291 | 141327582 | 291 | NM_006506 | RASA2 | | |
| 3 | 141328704 | 141328967 | 263 | NM_006506 | RASA2 | | |
| 3 | 141331097 | 141331283 | 186 | RASA2 | RASA2 | | |
| 3 | 141205943 | 141206114 | 171 | RASA2 | RASA2 | | |
| 3 | 141235149 | 141235382 | 233 | RASA2 | RASA2 | | |
| 3 | 141248526 | 141248704 | 178 | RASA2 | RASA2 | | |
| 3 | 141259339 | 141259533 | 194 | RASA2 | RASA2 | | |
| 3 | 141272657 | 141272823 | 166 | RASA2 | RASA2 | | |
| 3 | 141274563 | 141274760 | 197 | RASA2 | RASA2 | | |
| 3 | 141277701 | 141277869 | 168 | RASA2 | RASA2 | | |
| 3 | 141278667 | 141278854 | 187 | RASA2 | RASA2 | | |
| 3 | 141290219 | 141290406 | 187 | RASA2 | RASA2 | | |
| 3 | 141291424 | 141291602 | 178 | RASA2 | RASA2 | | |
| 3 | 141291956 | 141292181 | 225 | RASA2 | RASA2 | | |
| 3 | 141292778 | 141292956 | 178 | RASA2 | RASA2 | | |
| 3 | 141295800 | 141295981 | 181 | RASA2 | RASA2 | | |
| 3 | 141299182 | 141299373 | 191 | RASA2 | RASA2 | | |
| 3 | 141299852 | 141300039 | 187 | RASA2 | RASA2 | | |
| 3 | 141304821 | 141305023 | 202 | RASA2 | RASA2 | | |
| 3 | 141305477 | 141305728 | 251 | RASA2 | RASA2 | | |
| 3 | 141308900 | 141309125 | 225 | RASA2 | RASA2 | | |
| 3 | 141326474 | 141326643 | 169 | RASA2 | RASA2 | | |
| 3 | 141328162 | 141328374 | 212 | RASA2 | RASA2 | | |
| 13 | 48877974 | 48878244 | 271 | NM_000321 | RB1 | | |
| 13 | 48881369 | 48881595 | 227 | NM_000321 | RB1 | | |
| 13 | 48916681 | 48916905 | 225 | NM_000321 | RB1 | | |
| 13 | 48919205 | 48919392 | 188 | NM_000321 | RB1 | | |
| 13 | 48921889 | 48922104 | 216 | NM_000321 | RB1 | | |
| 13 | 48922996 | 48923282 | 287 | NM_000321 | RB1 | | |
| 13 | 48934078 | 48934352 | 275 | NM_000321 | RB1 | | |
| 13 | 48936909 | 48937131 | 223 | NM_000321 | RB1 | | |
| 13 | 48938921 | 48939120 | 200 | NM_000321 | RB1 | | |
| 13 | 48941538 | 48941753 | 216 | NM_000321 | RB1 | | |
| 13 | 48942568 | 48942790 | 223 | NM_000321 | RB1 | | |
| 13 | 48947426 | 48947649 | 224 | NM_000321 | RB1 | | |
| 13 | 48950981 | 48951241 | 261 | NM_000321 | RB1 | | |
| 13 | 48953640 | 48953798 | 159 | NM_000321 | RB1 | | |
| 13 | 48954259 | 48954413 | 155 | NM_000321 | RB1 | | |
| 13 | 48955327 | 48955589 | 263 | NM_000321 | RB1 | | |
| 13 | 49027073 | 49027292 | 220 | NM_000321 | RB1 | | |
| 13 | 49030298 | 49030538 | 241 | NM_000321 | RB1 | | |
| 13 | 49033803 | 49033997 | 195 | NM_000321 | RB1 | | |
| 13 | 49037808 | 49038047 | 240 | NM_000321 | RB1 | | |
| 13 | 49039099 | 49039526 | 428 | NM_000321 | RB1 | | |
| 13 | 49047361 | 49047640 | 280 | NM_000321 | RB1 | | |
| 13 | 49050803 | 49051051 | 249 | NM_000321 | RB1 | | |
| 13 | 49051413 | 49051603 | 191 | NM_000321 | RB1 | | |
| 13 | 49054028 | 49054249 | 222 | NM_000321 | RB1 | | |
| 3 | 47058523 | 47058803 | 280 | SETD2_E21 | SETD2 | | |
| 3 | 47059078 | 47059278 | 200 | SETD2_E20 | SETD2 | | |
| 3 | 47061189 | 47061389 | 200 | SETD2_E19 | SETD2 | | |
| 3 | 47079091 | 47079331 | 240 | SETD2_E18 | SETD2 | | |
| 3 | 47084000 | 47084240 | 240 | SETD2_E17 | SETD2 | | |
| 3 | 47087923 | 47088163 | 240 | SETD2_E16 | SETD2 | | |
| 3 | 47098285 | 47099005 | 720 | SETD2_E15 | SETD2 | | |
| 3 | 47103613 | 47103893 | 280 | SETD2_E14 | SETD2 | | |
| 3 | 47108503 | 47108663 | 160 | SETD2_E13 | SETD2 | | |
| 3 | 47125200 | 47125880 | 680 | SETD2_E12 | SETD2 | | |
| 3 | 47127624 | 47127864 | 240 | SETD2_E11 | SETD2 | | |
| 3 | 47129549 | 47129789 | 240 | SETD2_E10 | SETD2 | | |
| 3 | 47139387 | 47139627 | 240 | SETD2_E9 | SETD2 | | |
| 3 | 47142896 | 47143096 | 200 | SETD2_E8 | SETD2 | | |
| 3 | 47144774 | 47144974 | 200 | SETD2_E7 | SETD2 | | |
| 3 | 47147428 | 47147668 | 240 | SETD2_E6 | SETD2 | | |
| 3 | 47155315 | 47155555 | 240 | SETD2_E5 | SETD2 | | |
| 3 | 47158058 | 47158298 | 240 | SETD2_E4 | SETD2 | | |
| 3 | 47161654 | 47166054 | 4400 | SETD2_E3 | SETD2 | | |
| 3 | 47168045 | 47168245 | 200 | SETD2_E2 | SETD2 | | |
| 3 | 47205278 | 47205478 | 200 | SETD2_E1 | SETD2 | | |
| 2 | 198266392 | 198266632 | 241 | SF3B1_E16 | SF3B1 | | |
| 2 | 198266661 | 198266901 | 241 | SF3B1_E15 | SF3B1 | | |
| 2 | 198267254 | 198267574 | 321 | SF3B1_E14 | SF3B1 | | |
| 2 | 198267615 | 198267815 | 201 | SF3B1_E13 | SF3B1 | | |
| 2 | 198268258 | 198268538 | 281 | SF3B1_E12 | SF3B1 | | |
| 2 | 231090542 | 231090716 | 175 | NM_007237 | SP140 | | |
| 2 | 231101778 | 231102002 | 225 | NM_007237 | SP140 | | |
| 2 | 231102869 | 231103128 | 260 | NM_007237 | SP140 | | |
| 2 | 231103467 | 231103637 | 171 | NM_007237 | SP140 | | |
| 2 | 231106028 | 231106231 | 204 | NM_007237 | SP140 | | |
| 2 | 231108404 | 231108646 | 243 | NM_007237 | SP140 | | |
| 2 | 231109702 | 231109887 | 186 | NM_007237 | SP140 | | |
| 2 | 231110484 | 231110658 | 175 | NM_007237 | SP140 | | |
| 2 | 231112592 | 231112811 | 220 | NM_007237 | SP140 | | |
| 2 | 231113557 | 231113757 | 201 | NM_007237 | SP140 | | |
| 2 | 231115563 | 231115795 | 233 | NM_007237 | SP140 | | |
| 2 | 231118006 | 231118223 | 218 | NM_007237 | SP140 | | |
| 2 | 231120034 | 231120266 | 233 | NM_007237 | SP140 | | |
| 2 | 231134129 | 231134333 | 205 | NM_007237 | SP140 | | |
| 2 | 231134499 | 231134769 | 271 | NM_007237 | SP140 | | |
| 2 | 231135267 | 231135496 | 230 | NM_007237 | SP140 | | |
| 2 | 231148982 | 231149174 | 193 | NM_007237 | SP140 | | |
| 2 | 231150444 | 231150663 | 220 | NM_007237 | SP140 | | |
| 2 | 231152583 | 231152815 | 233 | NM_007237 | SP140 | | |
| 2 | 231155161 | 231155391 | 231 | NM_007237 | SP140 | | |
| 2 | 231157279 | 231157516 | 238 | NM_007237 | SP140 | | |
| 2 | 231158918 | 231159113 | 196 | NM_007237 | SP140 | | |
| 2 | 231162048 | 231162233 | 186 | NM_007237 | SP140 | | |
| 2 | 231174614 | 231174831 | 218 | NM_007237 | SP140 | | |
| 2 | 231175440 | 231175601 | 162 | NM_007237 | SP140 | | |
| 2 | 231175799 | 231175988 | 190 | NM_007237 | SP140 | | |
| 2 | 231176154 | 231176361 | 208 | NM_007237 | SP140 | | |
| 2 | 231177247 | 231177492 | 246 | NM_007237 | SP140 | | |
| 4 | 106155088 | 106158608 | 3521 | TET2_E3 | TET2 | | |
| 4 | 106162440 | 106162640 | 201 | TET2_E4 | TET2 | | |
| 4 | 106163937 | 106164137 | 201 | TET2_E5 | TET2 | | |
| 4 | 106164710 | 106164950 | 241 | TET2_E6 | TET2 | | |
| 4 | 106180710 | 106180990 | 281 | TET2_E7 | TET2 | | |
| 4 | 106182860 | 106183060 | 201 | TET2_E8 | TET2 | | |
| 4 | 106190715 | 106190955 | 241 | TET2_E9 | TET2 | | |
| 4 | 106193697 | 106194097 | 401 | TET2_E10 | TET2 | | |
| 4 | 106196051 | 106196251 | 201 | TET2_E11 | TET2 | | |
| 4 | 106196297 | 106197697 | 1401 | TET2_E11 | TET2 | | |
| 13 | 95226973 | 95227133 | 160 | NM_014305 | TGDS | | |
| 13 | 95233271 | 95233463 | 192 | NM_014305 | TGDS | | |
| 13 | 95243049 | 95243294 | 245 | NM_014305 | TGDS | | |
| 13 | 95228531 | 95228699 | 168 | TGDS | TGDS | | |
| 13 | 95229592 | 95229798 | 206 | TGDS | TGDS | | |
| 13 | 95230256 | 95230426 | 170 | TGDS | TGDS | | |
| 13 | 95230845 | 95231063 | 218 | TGDS | TGDS | | |
| 13 | 95232096 | 95232216 | 120 | TGDS | TGDS | | |
| 13 | 95232219 | 95232339 | 120 | TGDS | TGDS | | |
| 13 | 95235330 | 95235513 | 183 | TGDS | TGDS | | |
| 13 | 95244462 | 95244631 | 169 | TGDS | TGDS | | |
| 13 | 95246048 | 95246247 | 199 | TGDS | TGDS | | |
| 13 | 95248187 | 95248414 | 227 | TGDS | TGDS | | |
| 17 | 7565290 | 7565382 | 93 | ENST00000413465 | TP53 | | |
| 17 | 7572904 | 7573074 | 171 | NM_001276761 | TP53 | | |
| 17 | 7573896 | 7574111 | 216 | NM_001276761 | TP53 | | |
| 17 | 7576538 | 7576659 | 122 | NM_001276761 | TP53 | | |
| 17 | 7576798 | 7577263 | 466 | NM_001276761 | TP53 | | |
| 17 | 7577468 | 7577622 | 155 | NM_001276761 | TP53 | | |
| 17 | 7578084 | 7578611 | 528 | NM_001276761 | TP53 | | |
| 17 | 7579269 | 7579619 | 351 | NM_001276761 | TP53 | | |
| 17 | 7579624 | 7579959 | 336 | NM_001276761 | TP53 | | |
| 17 | 7580563 | 7580759 | 197 | NM_001276761 | TP53 | | |
| 14 | 103336434 | 103336936 | 503 | NM_145726 | TRAF3 | | |
| 14 | 103338200 | 103338427 | 228 | NM_145726 | TRAF3 | | |
| 14 | 103341878 | 103342175 | 298 | NM_145726 | TRAF3 | | |
| 14 | 103342649 | 103342907 | 259 | NM_145726 | TRAF3 | | |
| 14 | 103352405 | 103352610 | 206 | NM_145726 | TRAF3 | | |
| 14 | 103355798 | 103356036 | 239 | NM_145726 | TRAF3 | | |
| 14 | 103357634 | 103357832 | 199 | NM_145726 | TRAF3 | | |
| 14 | 103361299 | 103361520 | 222 | NM_145726 | TRAF3 | | |
| 14 | 103363588 | 103363830 | 243 | NM_145726 | TRAF3 | | |
| 14 | 103369488 | 103370286 | 799 | NM_145726 | TRAF3 | | |
| 14 | 103371430 | 103372181 | 752 | NM_145726 | TRAF3 | | |
| 16 | 72821042 | 72821602 | 561 | ZFHX3 | ZFHX3 | | |
| 16 | 72821642 | 72822002 | 361 | ZFHX3 | ZFHX3 | | |
| 16 | 72822037 | 72822557 | 521 | ZFHX3 | ZFHX3 | | |
| 16 | 72822567 | 72822847 | 281 | ZFHX3 | ZFHX3 | | |
| 16 | 72827130 | 72831370 | 4241 | ZFHX3 | ZFHX3 | | |
| 16 | 72831398 | 72832638 | 1241 | ZFHX3 | ZFHX3 | | |
| 16 | 72833856 | 72834096 | 241 | ZFHX3 | ZFHX3 | | |
| 16 | 72845455 | 72845695 | 241 | ZFHX3 | ZFHX3 | | |
| 16 | 72845750 | 72845990 | 241 | ZFHX3 | ZFHX3 | | |
| 16 | 72863617 | 72863817 | 201 | ZFHX3 | ZFHX3 | | |
| 16 | 72923605 | 72923885 | 281 | ZFHX3 | ZFHX3 | | |
| 16 | 72984355 | 72984875 | 521 | ZFHX3 | ZFHX3 | | |
| 16 | 72991304 | 72991704 | 401 | ZFHX3 | ZFHX3 | | |
| 16 | 72991714 | 72992594 | 881 | ZFHX3 | ZFHX3 | | |

The 69 genes set forth in the able above are: Actin Gamma 1(ACTG1), Protein kinase B (AKT1), Anaplastic Lymphoma Kinase (ALK), AT Rich Interactive Domain 1A (ARID1A), ASXL Transcriptional Regulator 1 (ASXL1), ASXL Transcriptional Regulator 3(ASXL3), Ataxia-Telangiectasia Mutated (ATM), Ataxia telangiectasia and Rad3 related (ATR), alpha-thalassemia/mental retardation, X-linked (ATRX), B-cell CLL/lymphoma 7 (BCL7A), B-Raf Proto-Oncogene, Serine/Threonine Kinase (BRAF), Cyclin D1 (CCND1), Cadherin-4 (CDH4), Cyclin-dependent kinase inhibitor 1B (CDKN1B), Cyclin Dependent Kinase Inhibitor 2C (CDKN2C), CREB-binding protein (CREBBP), Chr. C-X-C chemokine receptor type 4 (CXCR4), CYLD lysine 63 deubiquitinase (CYLD), Exosome complex exonuclease RRP44 (DIS3), DNA Methyltransferase 3 Alpha (DNMT3A), Early growth response protein 1(EGR1), E1A binding protein p300 (EP300), ETS translocation variant 4 (ETV4), Protein FAM46C (FAM46C), Fibroblast growth factor receptor 3 (FGFR3), Far Upstream Element Binding Protein 1 (FUBP1), HIST1H1C, HIST1H1E, HIST1H3G, HIST1H3H, Isocitrate Dehydrogenase 1 (IDH1), Isocitrate Dehydrogenase 2 (IDH2), Insulin-like Growth Factor 1 Receptor (IGF1R), Interferon Regulatory Factor 4 (IRF4), Lysine-Specific Demethylase 5C (KDM5C), Lysine-specific Demethylase 6A (KDM6A), Histone-lysine N-methyltransferase 2A (KMT2A), Lysine Methyltransferase 2B (KMT2B), Lysine Methyltransferase 2C (KMT2C), Lysine Methyltransferase 2D (KMT2D), Kirsten Rat Sarcoma (KRAS), Lymphotoxin-beta (LTB), MAF, MAFB, myc-associated factor X (MAX), Myeloid Differentiation Primary Response Protein (MYD88), Nuclear Receptor Corepressor 1 (NCOR1), Neurofibromin 1 (NF1), Nuclear Factor Of Kappa Light Polypeptide Gene Enhancer In B-Cells Inhibitor (NFKBIA), Neurogenic Locus Notch Homolog Protein 1 (NOTCH1), Neuroblastoma RAS (NRAS), NRM, Phosphatidylinositol-4,5-Bisphosphate 3-Kinase Catalytic Subunit Alpha (PIK3CA), Protein Phosphatase, Mg2+/Mn2+ Dependent 1D (PPM1D), PRAME Family Member 2 (PRAMEF2), PR Domain Zinc Finger Protein 1 (PRDM1), Serine/threonine-Protein Kinase D2 (PRKD2), Prune Homolog 2 With BCH Domain (PRUNE2), Protein-Tyrosine Phosphatase Non-Receptor Type 11 (PTPN11), RAS P21 Protein Activator 2 (RASA2), Retinoblastoma Associated Protein (RB1), SET Domain Containing 2, Histone Lysine Methyltransferase (SETD2), Splicing Factor 3b Subunit 1 (SF3B1), SP140, Ten-Eleven Translocation Methylcytosine Dioxygenase 2 (TET2), TDP-Glucose 4,6-Dehydratase (TGDS), Tumor Protein p53 (TP53), TNF Receptor Associated Factor 3 (TRAF3), and Zinc Finger Homeobox Protein 3 (ZFHX3).

A 5% cutoff was selected to only include SNVs that are driver events in MM or CHIP pathogenesis. The panel was designed to be of a small size (>0.5 megabases). The size and target selection of the panel allow higher sensitivity and target coverage and thus, better performance. The 69-gene panel was compared to a larger panel of 400 pan cancer genes based on the TCGA dataset. The two panels were compared on 12 myeloma tumor and normal samples. A higher median target coverage for the 69-gene panel compared to the larger one was identified (FIG-8). This characteristic of the panel ensures a higher sensitivity to detect these genetic alterations and a cost-effective approach to sequence at less depth.

The 314 kb, 69-gene targeted sequencing panel was developed, including the relevant analytical pipelines to suppress errors from sequencing. A high efficiency in hybrid selection (90%) was confirmed, and the benchmarking demonstrated high sensitivity to detect low allele fractions (i.e. >75% sensitivity to detect 0.2% VAF) with zero false positives across multiple replicates when starting with 20 ng of cell-free DNA input. For example, targets with greater than 10,000× coverage exhibit greater than 95% sensitivity to detect low level mutations with fewer than 2 false positives. High on target percent with an average of 95.5% was achieved.

This panel was benchmarked and applied to baseline cell-free DNA samples from 30 patients with SMM diagnosis. It was also applied to 15 newly diagnosed patients in a CLIA setting for future application and use in clinical settings and to report results to care providers.

The cfDNA and their matched normal samples were sequenced at a raw depth of 25,000×. Afterwards, bioinformatics analysis was used to achieve a duplex median target coverage (MTC) of 1201× and 729× in normal and cfDNA samples, respectively, compared to 910 and 650× in the larger pan cancer panel. The matching tumor samples from bone marrow aspirates were prepared for whole exome sequencing at 250× to be used as the ground truth for detecting the observed variants in cfDNA. The variants found in the bone marrows of the 15 newly diagnosed samples were detected in the cfDNA with the 69-gene panel.

For the purpose of MRD detection, it is more reasonable to increase the breadth of coverage of the somatic mutations in the tumor samples. To address this, the baitset design needs to be personalized, tailored to each person's genomic alterations, as they have been previously described by means of WES or WGS of bone marrow samples. The baitset comprises a standard backbone, targeting the curated list of genes, which following WGS of bone marrow samples, is enriched with CNAs and mutations. A computational pipeline necessary for the extraction of important alterations from WES and their addition to the baitset's standard backbone is also developed.

This method will allow for following of patient response to treatment and disease progression with their somatic mutations and CNAs, using cfDNA and CTCs derived from sequential peripheral blood plasma or urine samples.

The above-mentioned panel of 69 genes was tested in a panel of patient samples and analyzed. Also described is the personalization of the DTS baitset design for mutation detection, based on mutations detected through WGS, termed “mutation fingerprinting”. Mutation fingerprinting was then tested in a panel of patient samples and data analysis showed improved performance, compared to previous efforts. Accordingly, described herein is tumor fingerprinting as a method of MRD detection.

### Monoclonal Gammopathy of Undetermined Significance (MGUS)

Monoclonal Gammopathy of Undetermined Significance (MGUS) is considered to be a benign precursor condition that might progress to a lymphoproliferative disease or multiple myeloma. See, Lomas et al., 2020 Cancers, 12(6): 1554, incorporated herein by reference.

MGUS is characterized by the presence of a serum monoclonal paraprotein derived from immunoglobulin (Ig). MGUS may be classified into IgM and non-IgM MGUS, depending on the cellular clone responsible for the particular paraprotein. In most cases, IgM MGUS might develop into lymphoid malignancies, especially Waldenström's macroglobulinemia (WM), but also, rarely, other non-Hodgkin lymphomas such as chronic lymphocytic leukemia. Non-IgM MGUS is derived from mature plasma cells that might progress to multiple myeloma (MM).

Specifically, MGUS is diagnosed by identifying serum paraprotein<30 g/l (3 g/dl), clonal plasma cells<10% on bone marrow biopsy, and no myeloma-related organ or tissue impairment or a related B-cell lymphoproliferative disorder.

### Smoldering Multiple Myeloma (SMM)

Smoldering multiple myeloma (SMM) is an asymptomatic disorder of clonal plasma cells (PCs). See, Rajkumar et al., 2015, Blood, 125(20): 3069-3075, incorporated herein by reference. SMM is characterized by the presence of a serum monoclonal (M) protein (IgG or IgA) of ≥3 g/dL and/or clonal bone marrow PCs (BMPCs) 10% to 60% with no evidence of end-organ damage (e.g., calcium elevation, renal dysfunction, anemia, or bone disease (i.e., CRAB criteria) or other myeloma-defining events (MDE).

Baseline studies to diagnose SMM should include complete blood count, serum creatinine, serum calcium, skeletal survey, serum protein electrophoresis with immunofixation, 24-hour urine protein electrophoresis with immunofixation, and serum FLC assay. Specialized imaging, e.g., Magnetic Resonance Imaging (MRI) of the spine and pelvis or whole-body MRI is recommended to exclude MM. The complete blood count, creatinine, calcium, M protein, and serum FLC levels should be re-evaluated every 3 to 4 months.

The standard of care for SMM is careful observation until the development of symptomatic MM. However, treatment options using, e.g., thalidomide, zoledronic acid, lenalidomide, dexamethasone, ixazomib, elotuzomib, elotuzumab, daratumumab, and pomalidomide are being developed. See, Rajkumar et al., 2015, Blood, 125(20): 3069-3075, incorporated herein by reference.

### Multiple Myeloma (MM)

Multiple myeloma (MM) is a malignant condition characterized by the accumulation of clonally proliferating plasma cells (PCs) in bone marrow (BM), and is the second most common hematological neoplasm worldwide. The cancer cells accumulate in the bone marrow, where they crowd out healthy blood cells. Multiple myeloma is the second most common hematologic cancer, representing 1% of all cancer diagnoses and 2% of all cancer deaths. Despite recent progress in the management of patients, myeloma remains an incurable disease, with a median survival not exceeding 4 years.

Several characteristic genetic changes lead to the creation of a MM. These changes include chromosomal translocations, intrachromosomal rearrangements, single nucleotide variations (SNVs), copy number alterations (CNAs), chromosome translocation breakpoints, and variable, density, and joining (VDJ) rearrangement.

The most common signs and symptoms of MM can vary, and early stages of the disease does not manifest in symptoms. General symptoms can include bone pain, especially in the spine or chest, nausea, constipation, loss of appetite, mental fogginess or confusion, fatigue, frequent infections, weight loss, weakness or numbness in the legs, and excessive thirst.

MM is diagnosed through laboratory tests, such as urine analysis (e.g., screening for Bence Jones proteins), bone marrow biopsy, X-Ray and Magnetic Resonance Imaging (MRI). However, it most often diagnosed through a simple blood count test which screens for protein produced by the MM cells (e.g., beta-2-microglobulin or IgG/IgA antibodies).

Specifically, symptomatic multiple myeloma is diagnosed by identifying clonal plasma cells>10% on bone marrow biopsy or (in any quantity) in a biopsy from other tissues (plasmacytoma); a monoclonal protein (myeloma protein) in either serum or urine (except in cases of true nonsecretory myeloma); and evidence of end-organ damage felt related to the plasma cell disorder (related organ or tissue impairment, CRAB): HyperCalcemia (corrected calcium>2.75 mmol/1, >11 mg/dl), Renal failure (kidney insufficiency) attributable to myeloma, Anemia (hemoglobin<10 g/dl), and Bone lesions (lytic lesions or osteoporosis with compression fractures).

Because MM is complex and incurable, treatment is dependent on monitoring the progression of the disease. Standard treatments for MM include targeted therapy, biological therapy, chemotherapy, corticosterioids, radiation, and stem cell and bone marrow transplant.

Chemotherapy and radiation is the initial treatment of choice, and most people with MM receive a combination of medications. Exemplary agents include lenalidomide, dexamethasone, bortezomib, thalidomide, melphlan, vincristine, doxorubicin, etoposide, bendamustine or cyclophosphamide. Stem cell transplant, e.g., autologous or allogeneic hematopoietic stem cell transplantation, is also a preferred treatment for multiple myeloma.

### MRD

Minimal residual disease (MRD) refers to the small number of cancer cells that remain in the body after treatment. The number of remaining cells may be so small that they do not cause any physical signs or symptoms and often cannot even be detected through traditional methods, such as viewing cells under a microscope and/or by tracking abnormal serum proteins in the blood. An MRD positive test result means that residual (remaining) disease was detected. A negative result means that residual disease was not detected. MRD is used to measure the effectiveness of treatment and to predict which patients are at risk of relapse. It can also help confirm and monitor remissions, and possibly identify an early return of the cancer. Minimal residual disease may be present after treatment because not all of the cancer cells responded to the therapy, or because the cancer cells became resistant to the medications used.

To test for MRD, samples from either a blood draw or a bone marrow aspiration are used. For patients who are MRD positive, the number of remaining cancer cells may be so small that they cannot be detected through traditional tests, such as viewing cells under a microscope. The most widely used tests to measure MRD are flow cytometry, polymerase chain reaction (PCR) and next-generation sequencing (NGS).

Cell Free DNA (cfDNA)

Cell-free DNA (or cfDNA) refers to all non-encapsulated DNA in the blood stream. cfDNA are nucleic acid fragments that enter the bloodstream during cellular apoptosis or necrosis. Normally, these fragments are cleaned up by macrophages, but is overproduced by cancer cells. These fragments average around 170 bases in length, have a half-life of about two hours, and are present in both early and late stage disease in many common tumors. cfDNA concentration varies greatly, occurring at between 1 and 100,000 fragments per millilitres of plasma.

### Circulating Tumor Cells (CTC)

Circulating tumor cells (CTCs) are a rare subset of cells found in the blood of patients with solid tumors, which function as a seed for metastases. Cancer cells metastasize through the bloodstream either as single migratory CTCs or as multicellular groupings—CTC clusters. The CTCs preserve primary tumor heterogeneity and mimic tumor properties, and may be considered as clinical biomarker, preclinical model, and therapeutic target. The potential clinical application of CTCs is being a component of liquid biopsy. CTCs are also good candidates for generating preclinical models, especially 3D organoid cultures, which could be applied in drug screening, disease modeling, genome editing, tumor immunity, and organoid biobanks.

### Gene Expression Profiling

In general, methods of gene expression profiling may be divided into two large groups: methods based on hybridization analysis of polynucleotides and methods based on sequencing of polynucleotides. Methods known in the art for the quantification of mRNA expression in a sample include northern blotting and in situ hybridization, RNAse protection assays, RNA-seq, and reverse transcription polymerase chain reaction (RT-PCR). Alternatively, antibodies are employed that recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). For example, RT-PCR is used to compare mRNA levels in different sample populations, in normal and tumor tissues, with or without drug treatment, to characterize patterns of gene expression, to discriminate between closely related mRNAs, and/or to analyze RNA structure.

In some cases, a first step in gene expression profiling by RT-PCR is the reverse transcription of the RNA template into cDNA, followed by amplification in a PCR reaction. For example, extracted RNA is reverse-transcribed using a GeneAmp RNA PCR kit (Perkin Elmer, Calif, USA), following the manufacturer's instructions. The cDNA is then used as template in a subsequent PCR amplification and quantitative analysis using, for example, a TaqMan RTM (Life Technologies, Inc., Grand Island, N.Y.) assay.

### Next Generation Sequencing

In some embodiments, the somatic aberrations of MRD is determined by next-generation sequencing (NGS). These methods share the common feature of massively parallel, high-throughput strategies at relatively low lower costs compared to older sequencing methods. As known in the art, NGS methods can be broadly divided into those that typically use template amplification and those that do not. Amplification-requiring methods include pyrosequencing (commercially available from Roche as the 454 technology platforms (e.g., GS 20 and GS FLX)), the Solexa platform (commercially available from ILLUMINA™), and the Supported Oligonucleotide Ligation and Detection™ (SOLiD) platform (commercially available from APPLIED BIOSYSTEMS™. Non-amplification approaches, also known as single-molecule sequencing, may also be used. Examples include the HELISCOPE™ platform (commercially available from HELICOS BIOSYSTEMS™, and newer, real-time platforms (e.g., commercially available from VISIGEN™, OXFORD NANOPORE TECHNOLOGIES LTD., and PACIFIC BIOSCIENCES™).

### Whole Exome Sequencing (WES)

Whole-exome sequencing is a widely used next-generation sequencing (NGS) method that involves sequencing the protein-coding regions of the genome. The human exome represents less than 2% of the genome, but contains ˜85% of known disease-related variants, making this method a cost-effective alternative to whole-genome sequencing. Sequencing only the coding regions of the genome provides a focus on the genes most likely to affect phenotype. Exome sequencing detects variants in coding exons, with the capability to expand targeted content to include untranslated regions (UTRs) and microRNA for a more comprehensive view of gene regulation. DNA libraries can be prepared in as little as 1 day and require only 4-5 Gb of sequencing per exome.

### Deep Targeted Sequencing

Deep sequencing refers to sequencing a genomic region multiple times, sometimes hundreds or even thousands of times. Targeted gene sequencing panels are useful tools for analyzing specific mutations in a given sample. Focused panels contain a select set of genes or gene regions that have known or suspected associations with the disease or phenotype under study. Gene panels can be purchased with preselected content or custom designed to include genomic regions of interest. Deep sequencing is useful for studies in oncology, microbial genomics, and other research involving analysis of rare cell populations. For example, deep sequencing is required to identify mutations within tumors, because normal cell contamination is common in cancer samples, and the tumors themselves likely contain multiple sub-clones of cancer cells.

Described in detail below are the results from liquid biopsy assays in multiple myeloma.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: Characterization of Somatic Aberrations in Cell Free DNA (cfDNA) and Circulating Tumor Cells (CTCs) and their Utilization as Biomarkers of Progression in MGUS/SMM

cfDNA or CTC sequencing can be challenging because of i) the small fragment size of cfDNA in the peripheral blood (around 166 bp); ii) the low yield of DNA; and iii) the usual low fraction of tumor-derived DNA. Therefore, described herein are three different approaches to sequence cfDNA and CTC (Manier et al., Nature Communication, 2018. 9:1691, incorporated herein by reference (8).

Previously, the largest genomic profiling of 214 SMM patients identified high-risk genomic biomarkers associated with progression of SMM to MM (Bustoros et al., Journal of Clinical Oncology, 2020) (4).

### Methods

Next generation sequencing technologies were used to study 214 patients with SMM at time of diagnosis with a total of 223 samples including 5 serial samples. Whole exome sequencing (WES) was performed on 72 matched tumor-normal samples (mean target coverage 109X). WES was performed on 94 tumor-only samples (with mean coverage 174X), and targeted deep sequencing was performed on 48 samples (mean target coverage 774X). Samples were collected at Dana-Farber Cancer Institute, University College London, Mayo Clinic, and the University of Athens in Greece, in addition to multiple centers in the US and Europe. For 4 cases, serial samples were obtained at time of SMM diagnosis and time of progression to MM. One (1) case, who has not progressed to date, was sampled twice at the SMM stage. Samples were obtained after written informed consent, according to the Declaration of Helsinki. A subcohort of SMM patients who did not participate in any clinical trial (n=85) was examined to assess the natural history of disease progression.

### Results

Immunoglobulin heavy chain (IgH) translocations commonly seen in MM were present in 76 patients (36%), as identified by Fluorescence in Situ Hybridization (FISH), while SCNAs were the most common genomic alterations, and were present in 189 patients (88%). Hyperdiploidy (HRD), i.e., with 48 or more chromosomes in the genome, was found in 55% of patients; hypodiploidy, defined as less than 45 chromosomes, was found in only 10 patients (4.6%), and whole genome doubling (ploidy>2.5) in six (2.8%). The median mutation density in SMM patients was 1.4 mutation/Mb, and single nucleotide variations (SNVs) in genes significantly mutated in MM were present in 118 patient samples (55%). Forty-six percent of those had alterations in the MAPK pathway (KRAS, NRAS, BRAF, and PTPN11). DNA repair pathway alterations (TP53 and ATM SNVs and deletion 17p) were found in 21 (10%). SNVs in genes of NFkB, protein processing, and cell cycle pathways were found in 22%, 21%, and 6.7% patients, respectively. Bi-allelic inactivation events affecting TP53, RB1, CDKN2C, ZNF292, DIS3, or FAM46C were present in only 6% patients.

### Identifying Genomic Biomarkers of Progression

In the subgroup of 85 patients, the median follow-up time for all patients was 6.2 years. Median time to progression (TTP) was 3.9 years. In this cohort, 53 patients (62%) have progressed, while 32 (38%) remained asymptomatic.

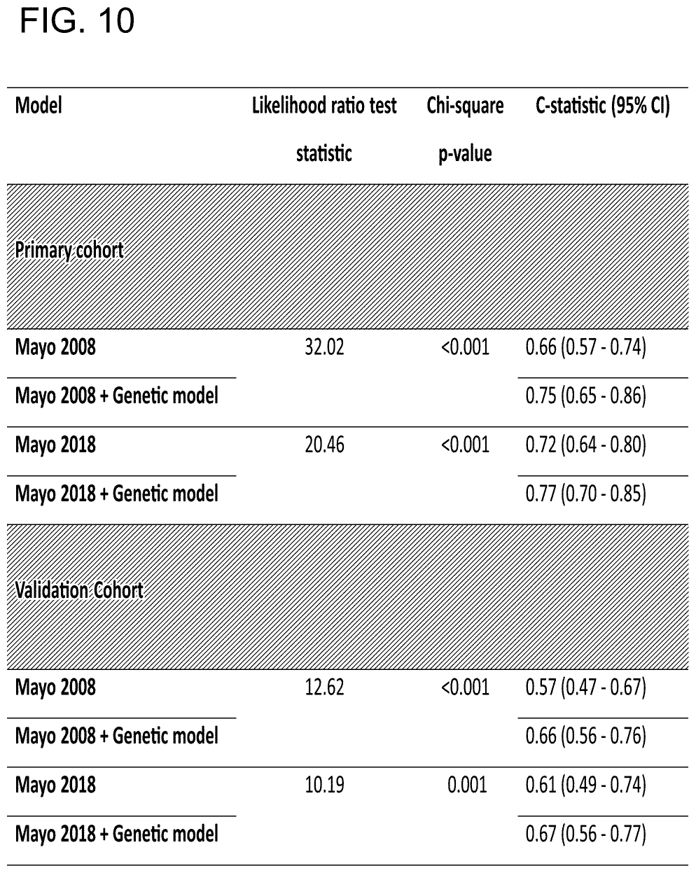

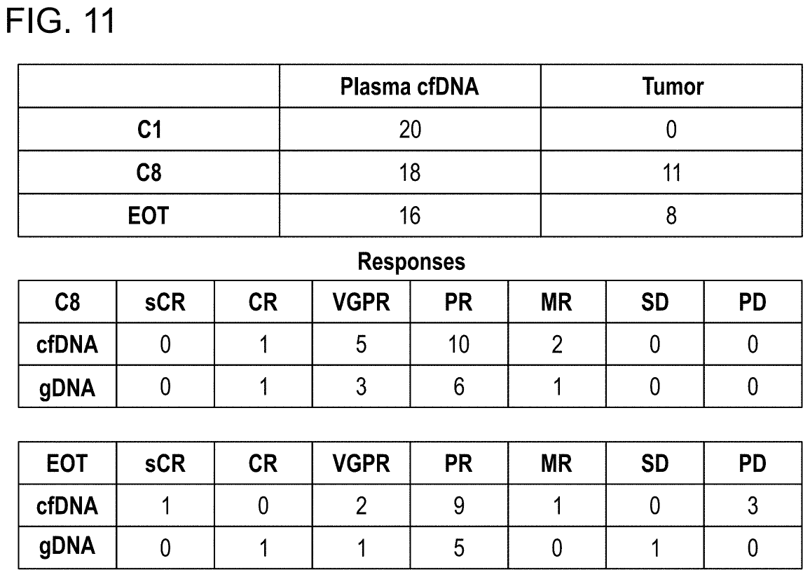

It was found that alterations in genes of the MAPK pathway (KRAS and NRAS SNVs), the DNA repair pathway (deletion 17p, TP53 and ATM SNVs), and MYC oncogene (translocations or CNVs) were all independent risk factors of progression and considered a high-risk genomic biomarkers after accounting for clinical risk staging (4). Thus a genomic risk score was developed based upon these three genomic alterations (GA). Of note, these results are independent of the clinical model used, whether it is Mayo 2008 or 2018 models. Interestingly, high-risk GA were found in patients described as low risk by both models, in whom they conferred a significantly increased risk of progression. Importantly, the genomic model improved the prediction of progression when added to the Mayo 2008 or 2018 models (p<0.001, C-statistic: 0.66 vs 0.75 and 0.72 vs 0.77, respectively) (FIG. 10).

To test the robustness and generalizability of the model, it was validated in an external cohort of 72 patients with SMM. It was found that patients with any of the high-risk genomic biomarkers (n=47) had a higher risk of progression (2.5 vs. 10 years, p=0.001). Importantly, in a multivariate analysis accounting for clinical risk group in this cohort, the genomic model was an independent risk factor of progression; when combined with the clinical model for SMM, the genomic model performed better than the clinical model alone (p<0.001). (C-statistic: 0.61 vs 0.67). This panel could be a companion to this new genomic risk score to help identify high-risk SMM patients who will progress in a short period and need therapeutic intervention before end-organ damage. The invention described herein has the advantage of being using blood and tissue samples instead of bone marrow aspirates in clinical settings.

### Example 2: Applying Ultra-Low Pass Whole Genome Sequencing (ULP-WGS) to Sequence cfDNA and CTC

### Methods

A minimum DNA concentration of 5 ng from cfDNA and CTC was subjected to library preparation using the Kapa HyperPlus kit and large numbers of cfDNA and CTC libraries were multiplexed and sequenced to an average of 0.1× genome-wide sequencing coverage. The statistical approach from the HMM copy software was applied to correct for GC-content and mappability (sequence uniqueness) biases in read counts within genomic bins of 1 Mb, which substantially improved signal to noise ratio. A modified approach was developed from the TITAN framework to perform segmentation, CNV prediction, and purity and ploidy estimation (called ichorCNA). The detectability of cfDNA and CTCs in blood samples from 107 and 56 patients with MM using ULP-WGS was examined. Plasma samples were isolated from whole blood EDTA tubes after two-step centrifugation: 300×g for 10 min and 3000×g for 10 min. DNA was extracted using Qiagen circulating nucleic acid kits from 2 to 6 mL of plasma. CTCs and bone marrow plasma cells were isolated using CD138 bead selection after Ficoll of whole blood and bone marrow samples, respectively. Peripheral blood mononuclear cell (PBMC) negative fractions were used for germline DNA. Genomic DNA was extracted using Qiagen DNA extraction kit. For ULP-WGS, libraries were prepared using the Kapa Hyper Prep kit with custom adapters (IDT and Broad Institute) starting with 5 ng of DNA.

Up to 96 libraries were pooled and sequenced using 100 bp paired-end runs over 1 lane on a HiSeq2500 (Illumina). For WES, libraries were prepared using the Kapa Hyper Prep kit with custom adapters (IDT and Broad Institute) starting with 20 ng of DNA. Libraries were then quantified using the PicoGreen (Life Technologies) and pooled up to 12-plex. Hybrid capture of cfDNA libraries was performed using the Nextera Rapid Capture Exome kit (Illumina) with custom blocking oligos (IDT and Broad Institute). Sequencing was performed using 100 bp paired-end runs on Illumina HiSeq4000 in high-output mode with two to four libraries per lane.

### Results

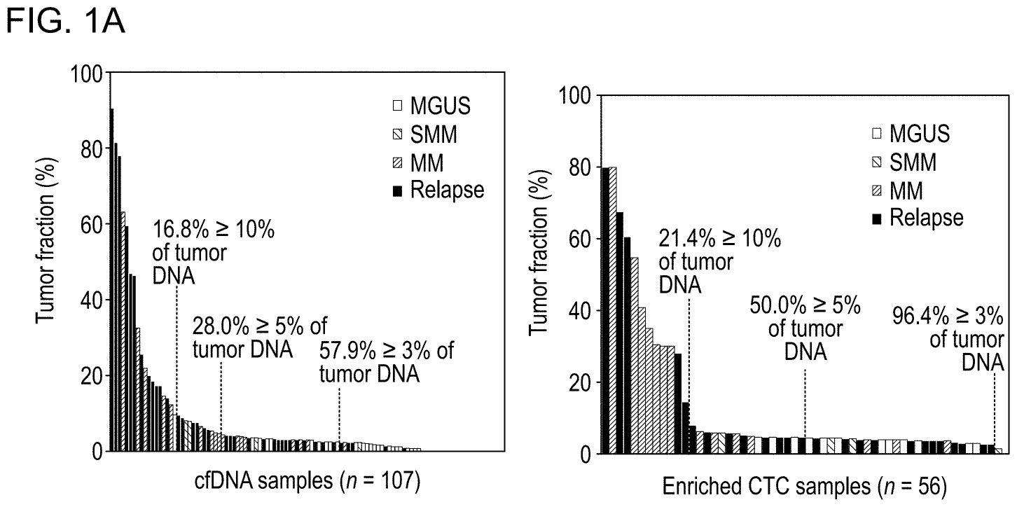

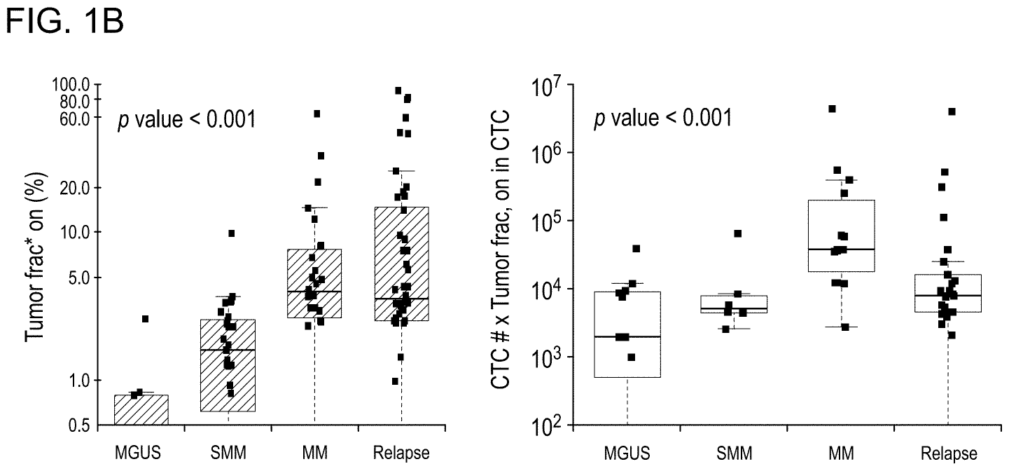

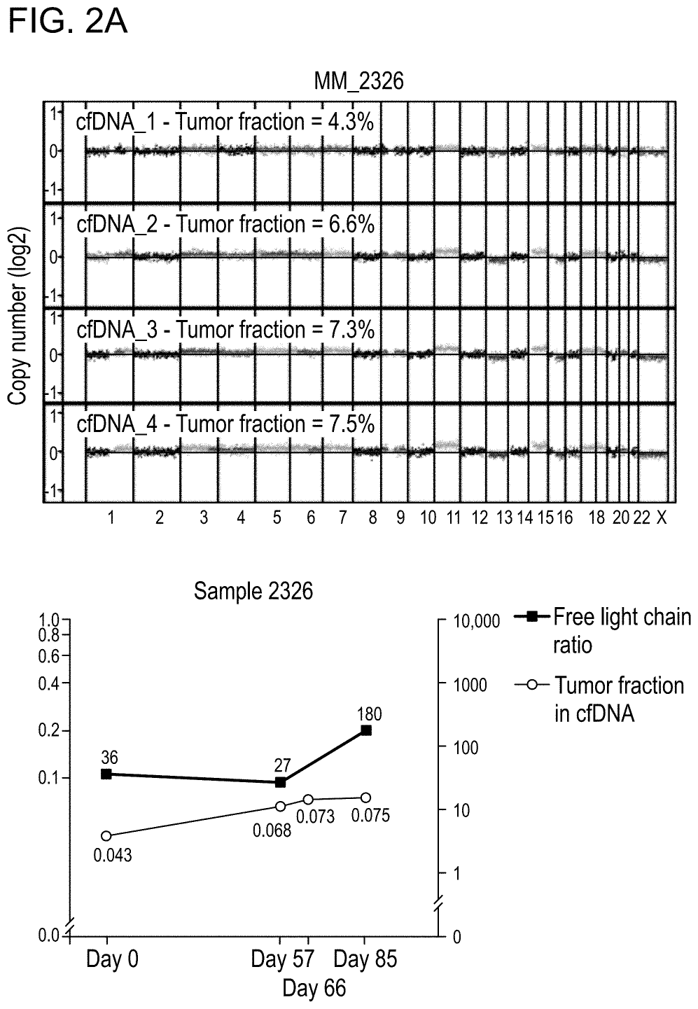

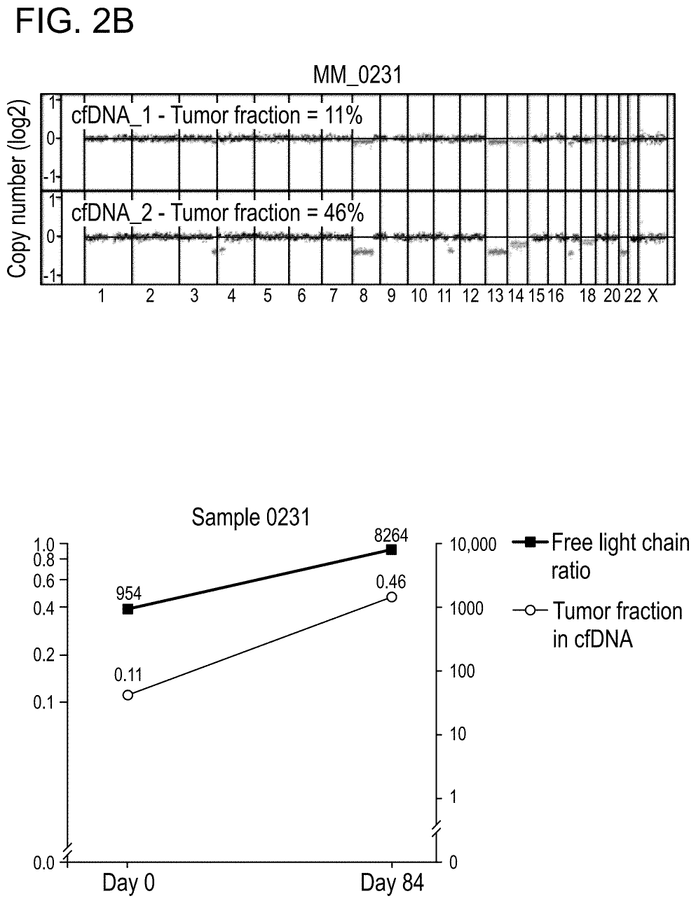

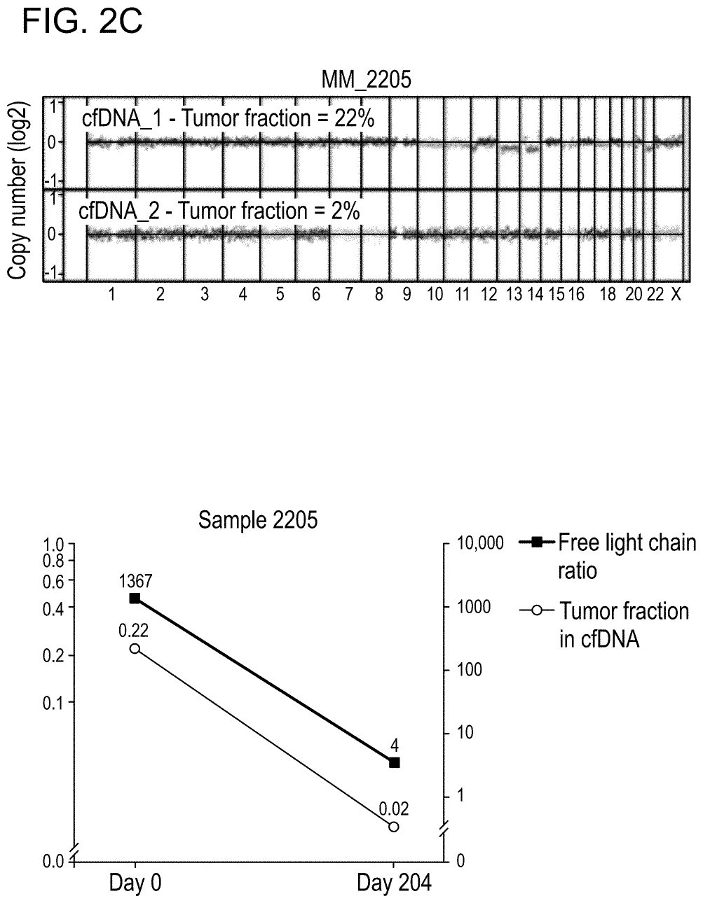

The data suggested that a significant fraction of patients with MM harbor detectable CTCs or cfDNA and that analyzing both cfDNA and CTCs may broaden the applicability of liquid biopsies to patients with MM. Among 70 cfDNA and 39 CTC samples of overt myeloma samples (newly diagnosed or relapsed), there was 76%, 41%, and 24% of cfDNA samples with ≥3, 5, and 10% tumor fraction, respectively. In comparison, there was 100%, 62%, and 31% of CTC samples having ≥3, 5, and 10% tumor fraction, respectively. Together, these data indicated that 76% and 100% of cfDNA and CTC samples, respectively, had a tumor fraction above 3%, the lower limit of detection of ichorCNA as previously benchmarked (Adalsteinsson et al., Nature Communications 2018). Interestingly, tumor fraction in cfDNA and CTCs (number of enriched CTC×tumor fraction) was significantly associated with the clinical stage of the disease. (FIG. 1A-FIG. 1B).

### Example 3: Applying Whole-Exome Sequencing (WES) to cfDNA, CTCs, and BM to Sequence cfDNA and CTC

### Methods

To assess whether cfDNA or CTCs or both can capture the genetic diversity of MM, WES was performed on matched cfDNA, CTCs and BM of 14 MM patients. Libraries were prepared and hybrid captured using the Nextera Rapid Capture Exome kit (Illumina) with 25 ng of DNA input.

Sequencing was performed on Illumina HiSeq4000 in high-output mode with 100 bp paired-end reads. Two to four libraries were pooled per lane.

### Results

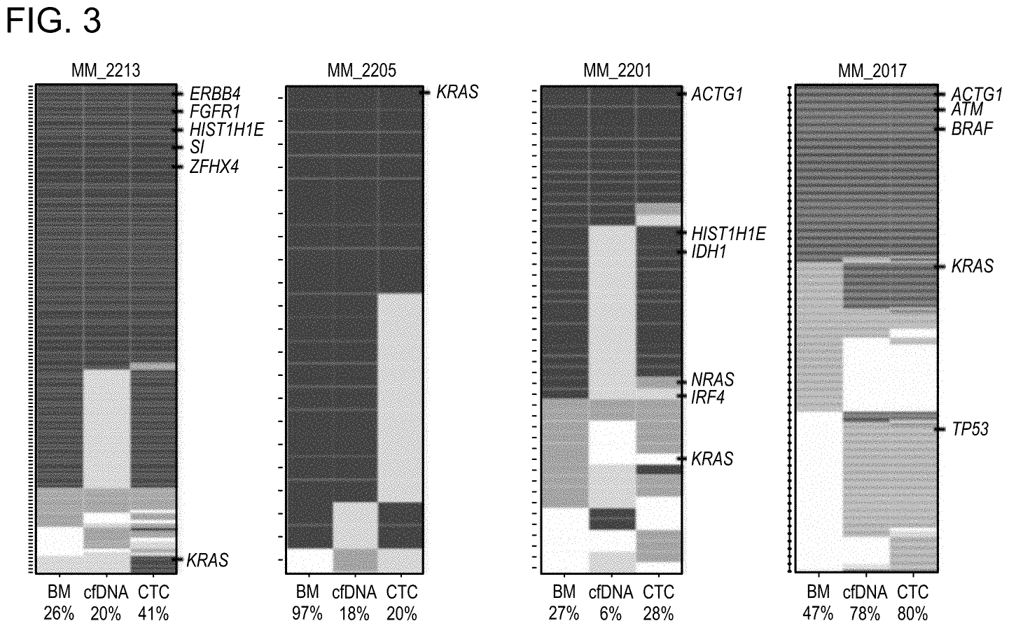

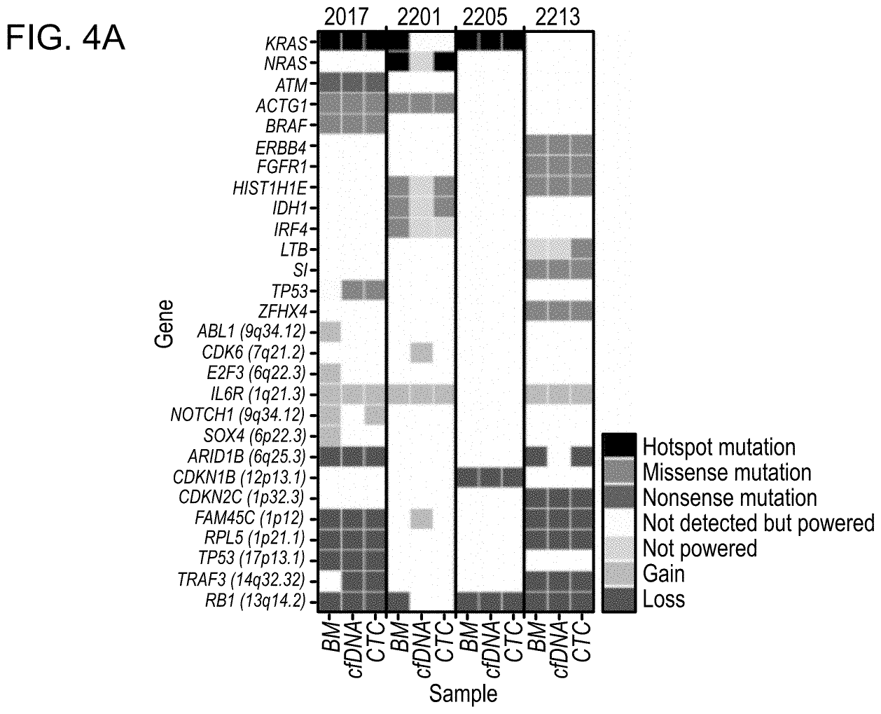

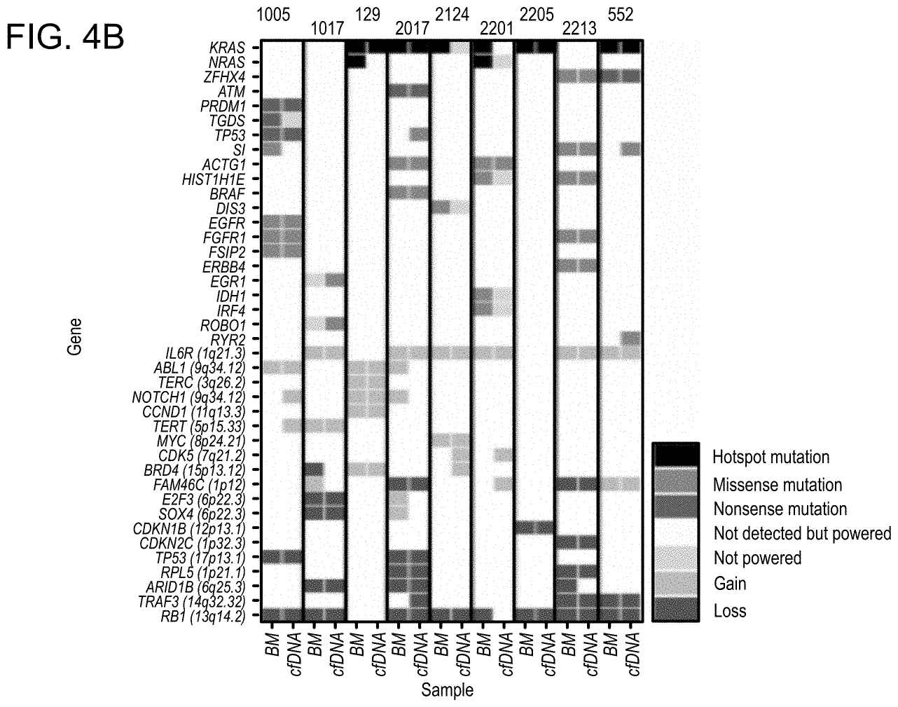

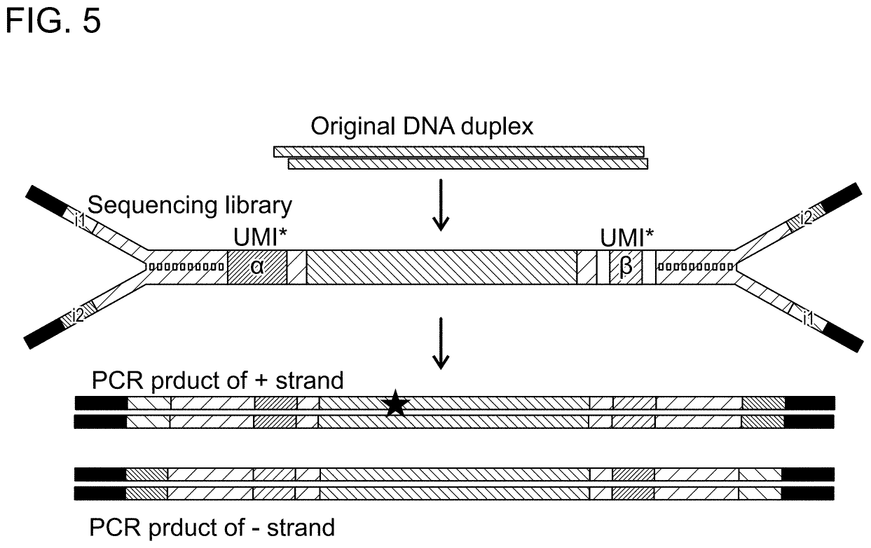

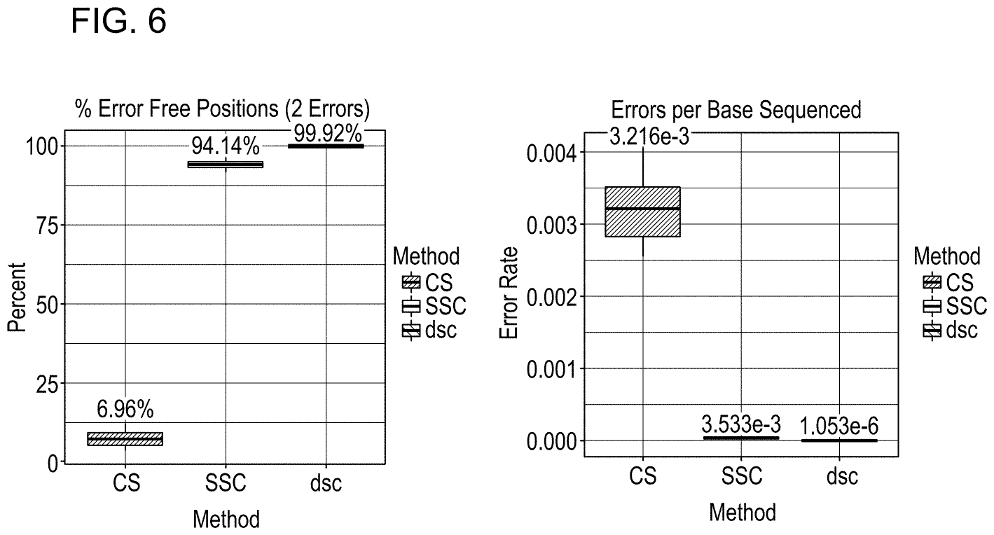

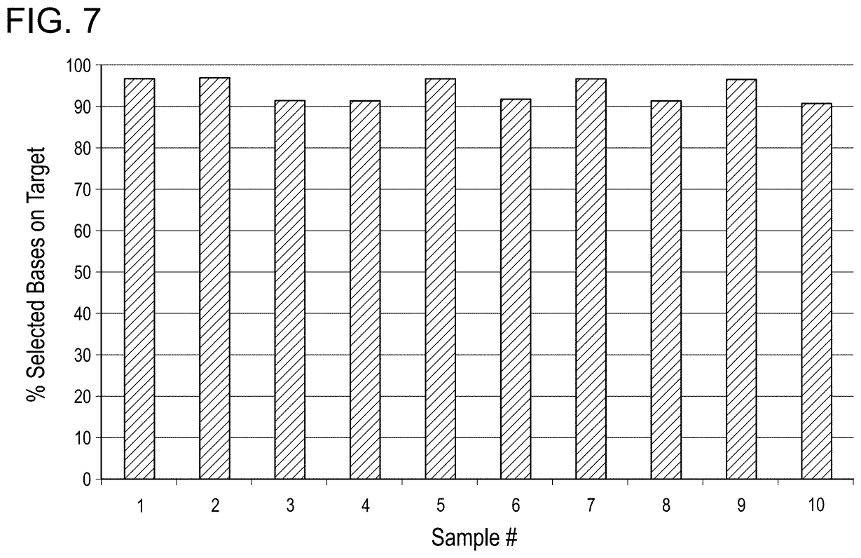

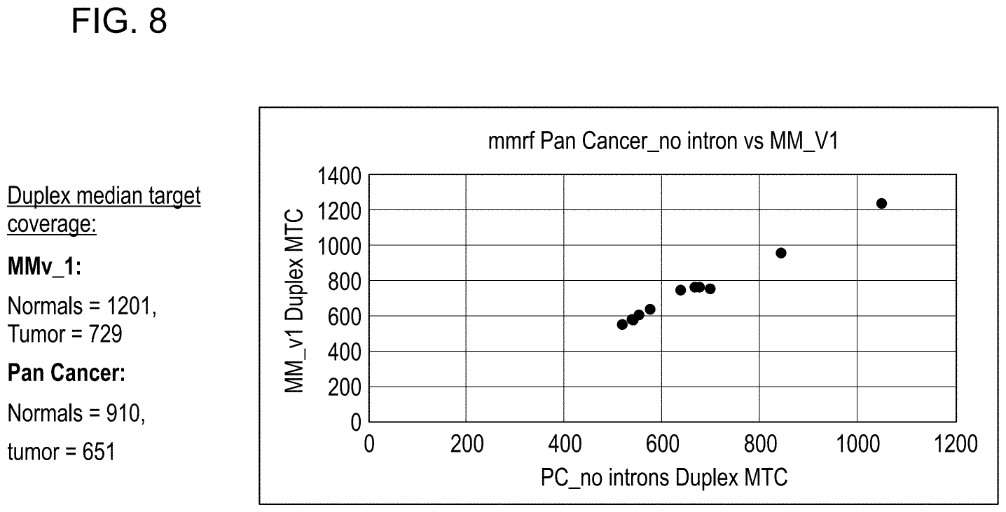

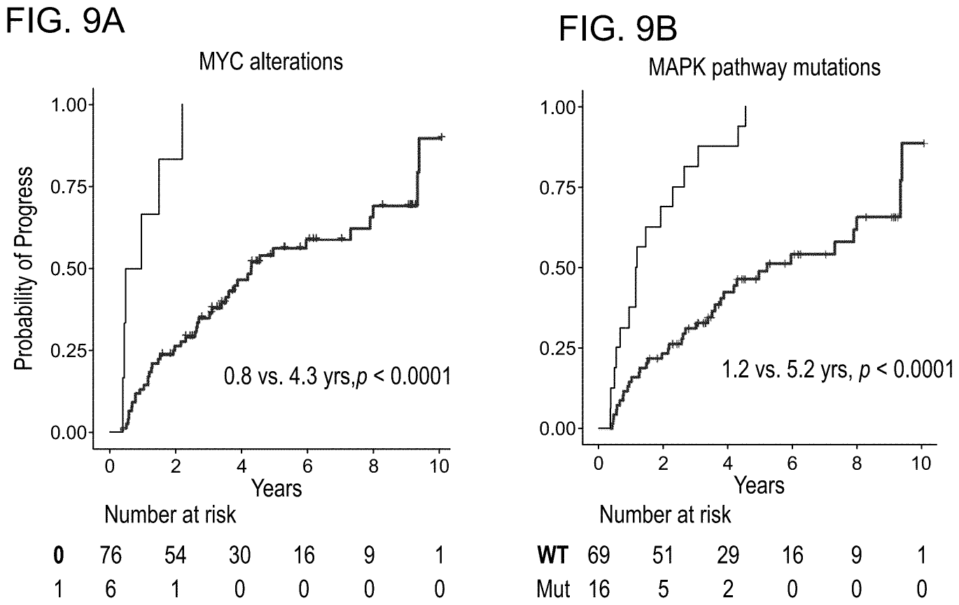

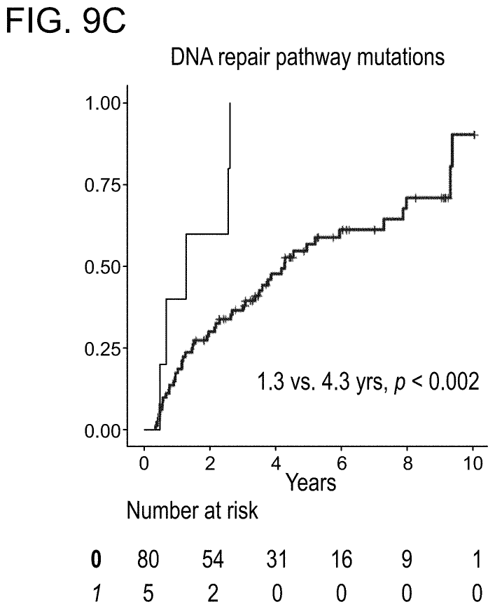

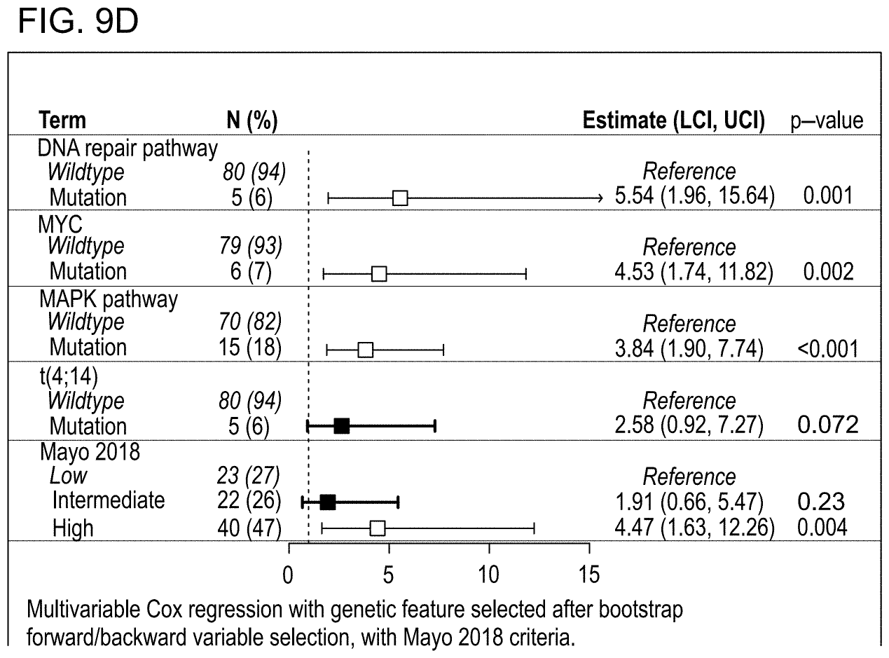

By comparing matched cfDNA/BM tumor DNA samples, a strong concordance was identified between three compartments in terms of CNAs and SNVs. Most interestingly, the combination of CTCs and cfDNA were able to detect almost all clonal mutations identified in the BM biopsy sample, including most recurrently mutated genes in MM (KRAS, NRAS, BRAF and TP53), and defined other subclones that were not identified in the bone marrow (FIG. 3 and FIG. 4).

### Example 4: Applying a Redesigned Capture Panel to Determine Minimal Residual Disease (MRD) Status and how Patient Tumor Mutations Change Over Time

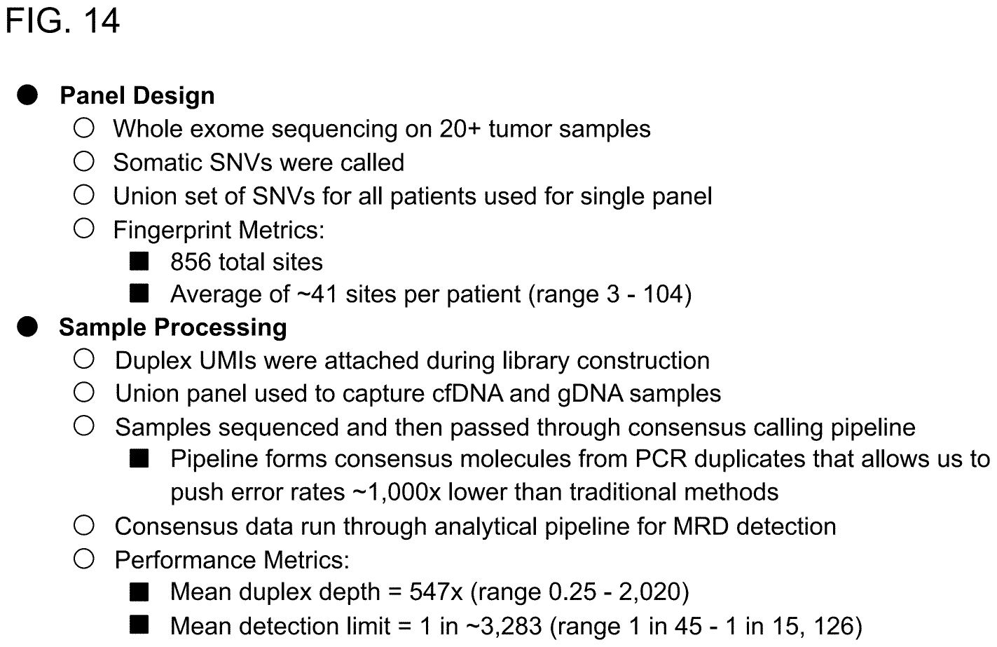

A personal capture panel was redesigned specifically for SMM patients as a fingerprint to study how the mutations from patient tumor biopsies change in blood over time. Specifically, a targeted gene panel was created encompassing all mutations identified via whole-exome sequencing of all eligible patients (n=20) in an investigator initiated phase II clinical trial using elotuzumab, lenalidomide and dexamethasone in SMM patients (FIG. 14).

### Methods

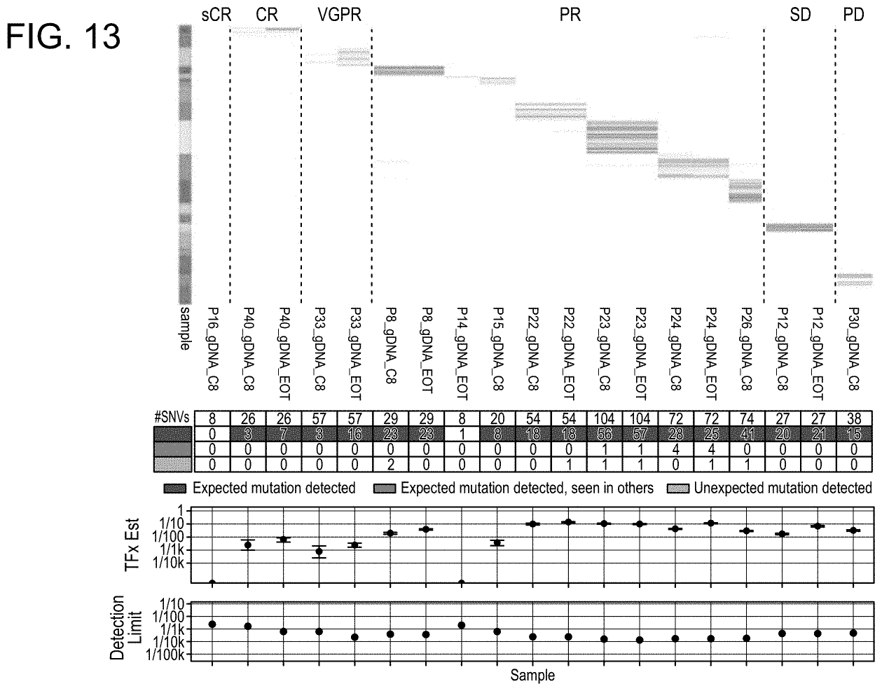

Using whole exome sequencing of the baseline bone marrow biopsy, somatic SNVs were discovered for each patient and aggregated them into a single individualized panel design. Then, 54 plasma cfDNA samples were identified for testing from these 20 patients, which were collected at baseline (n=20), end cycle 8 of treatment (n=18) and at the end of treatment protocol (n=16). The individualized panel was applied to all cfDNA sequencing libraries containing duplex UMI barcodes, which allowed the formation of consensus DNA duplexes after sequencing and implement error suppression methods that can reduce error rates ˜1,000× over traditional sequencing. To further suppress potential errors, any sites that showed mutant signal in samples in which that site was not specific were excluded from analysis. This final panel design included a total of 849 SNVs and a median of 34 SNVs (range 3-104) specific to each patient. A mean duplex depth of 560× (range 1×-1,882×) was achieved across all sites for each sample. First, it was determined whether it was possible to detect previously profiled somatic SNVs in baseline plasma cfDNA samples. Those plasma samples taken at baseline, prior to cycle one of treatment, were selected, and duplex consensus read pileups were created at each site in the panel (FIG. 13). Mutant signal was required from at least two distinct patient-specific sites to consider circulating tumor DNA (ctDNA) was detected.

### Results

Out of 20 patients with a baseline plasma cfDNA sample available, 12 patients had detectable ctDNA. Of those patients with detectable ctDNA, a median of 4 (range 2-57) patient-specific sites were detected. Using the number of specific sites tracked for a given patient sample and the number of mutant molecules recovered at each site, tumor fractions were estimated for samples with detectable ctDNA. Median estimated tumor fractions for samples with detectable ctDNA was 6.65e-4 (range 3.88e-5-9.78e-3). Notably, this was lower than benchmarking estimates for lower limits of detection using the multiple myeloma gene panel with 75% sensitivity. Also, it was determined whether if ctDNA could be detected in later time points throughout treatment as well. The same analysis was performed at the cycle 8 and end of treatment time points, and ctDNA was detected in 6 of 18 samples and 7 of 16 samples, respectively. These results suggest that using an individualized approach to detect minimal disease burden can increase our sensitivity over fixed gene panel approaches.

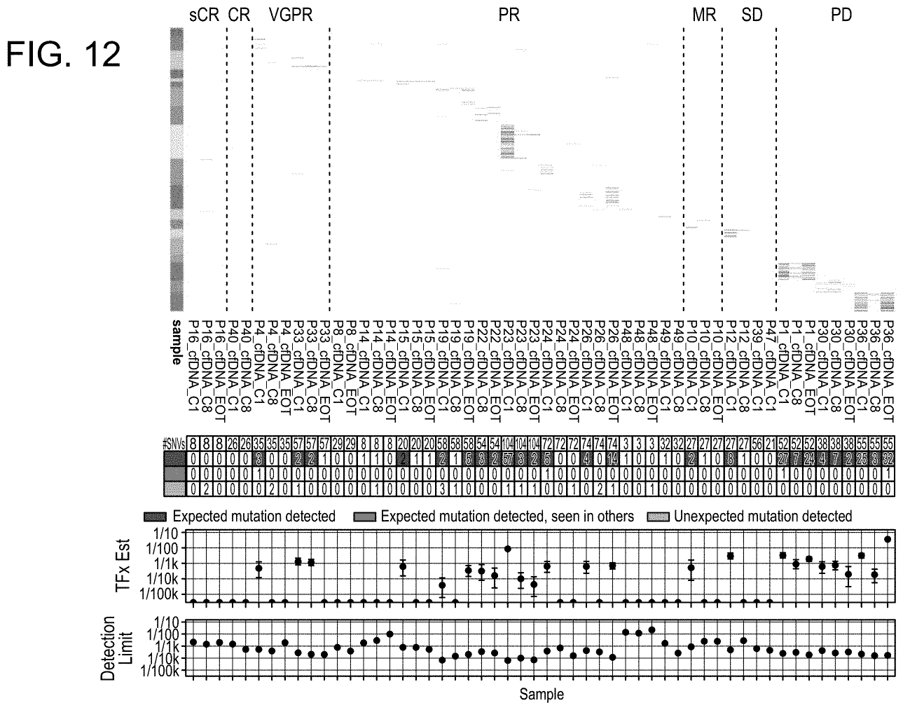

Given that ctDNA was detected in plasma samples across a range of tumor fractions, it was next determined whether there was a correlation between tumor fractions and response to treatment. First, tumor fractions estimated from baseline plasma samples were examined and compared to patients' response measured at the end of treatment and found tumor DNA fingerprint in all but 2 cfDNA samples (FIG. 12). Then, it was determined whether the detection of ctDNA at later time points could predict progression. It was reasoned that patients with detectable ctDNA during or after treatment may be at an increased risk for progression. As before, each sample was classified as having detectable ctDNA if two or more sites showed mutant signal, and a slight correlation was identified between ctDNA status, both at cycle 8 and end of treatment. Indeed, it was identified that patients with detectable ctDNA (MRD+ve) at Cycle 8 had shorter time to biochemical progression than those with no detectable ctDNA (p-value=0.046). The same observation was seen in EOT samples, where patients with detectable ctDNA had a trend of worse TTP compared to those who don't with borderline significant result (p-value=0.05), that could be explained by the smaller number of samples tested. In conclusion, this suggests that this individualized approach could potentially help identify which patients may be at highest risk for progression.

### REFERENCES

The following references were cited herein.
1. Weiss B M, Abadie J, Verma P, Howard R S, Kuehl W M. A monoclonal gammopathy precedes multiple myeloma in most patients. Blood. 2009; 113(22):5418-22.
2. Debes-Marun C S, Dewald G W, Bryant S, Picken E, Santana-Davila R, Gonzalez-Paz N, et al. Chromosome abnormalities clustering and its implications for pathogenesis and prognosis in myeloma. Leukemia. 2003; 17(2):427-36.
3. Kyle R A, Remstein E D, Therneau T M, Dispenzieri A, Kurtin P J, Hodnefield J M, et al. Clinical course and prognosis of smoldering (asymptomatic) multiple myeloma. N Engl J Med. 2007; 356(25):2582-90.
4. Bustoros M, Sklavenitis-Pistofidis R, Park J, Redd R, Zhitomirsky B, Dunford A J, et al. Genomic Profiling of Smoldering Multiple Myeloma Identifies Patients at a High Risk of Disease Progression. Journal of clinical oncology: official journal of the American Society of Clinical Oncology. 2020:Jco2000437.
5. Lohr J G, Stojanov P, Carter S L, Cruz-Gordillo P, Lawrence M S, Auclair D, et al. Widespread genetic heterogeneity in multiple myeloma: implications for targeted therapy. Cancer Cell. 2014; 25(1):91-101.
6. Walker B A, Boyle E M, Wardell C P, Murison A, Begum D B, Dahir N M, et al. Mutational Spectrum, Copy Number Changes, and Outcome: Results of a Sequencing Study of Patients With Newly Diagnosed Myeloma. J Clin Oncol. 2015; 33(33):3911-20.
7. Walker B A, Mavrommatis K, Wardell C P, Ashby T C, Bauer M, Davies F E, et al. Identification of novel mutational drivers reveals oncogene dependencies in multiple myeloma. Blood. 2018; 132(6):587-97.
8. Lindsley R C, Saber W, Mar B G, Redd R, Wang T, Haagenson M D, et al. Prognostic Mutations in Myelodysplastic Syndrome after Stem-Cell Transplantation. N Engl J Med. 2017; 376(6):536-47.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of determining whether a subject with monoclonal gammopathy of undetermined significance (MGUS) or smoldering multiple myeloma (SMM) will progress to multiple myeloma (MM) in a subject comprising:
obtaining a test sample from a subject having MGUS, SMM, or at risk of developing MM;
detecting a somatic aberration in at least one MM-associated gene in the test sample as compared to the MM-associated gene in a reference sample; and
determining that the subject will progress to MM.

2. The method of claim 1, wherein the at least one MRD-associated gene comprises at least one of Actin Gamma 1 (ACTG1), Protein kinase B (AKT1), Anaplastic Lymphoma Kinase (ALK), AT Rich Interactive Domain 1A (ARID1A), ASXL Transcriptional Regulator 1 (ASXL1), ASXL Transcriptional Regulator 3 (ASXL3), Ataxia-Telangiectasia Mutated (ATM), Ataxia telangiectasia and Rad3 related (ATR), alpha-thalassemia/mental retardation, X-linked (ATRX), B-cell CLL/lymphoma 7 (BCL7A), B-Raf Proto-Oncogene, Serine/Threonine Kinase (BRAF), Cyclin D1 (CCND1), Cadherin-4 (CDH4), Cyclin-dependent kinase inhibitor 1B (CDKN1B), Cyclin Dependent Kinase Inhibitor 2C (CDKN2C), CREB-binding protein (CREBBP), Chr. C-X-C chemokine receptor type 4 (CXCR4), CYLD lysine 63 deubiquitinase (CYLD), Exosome complex exonuclease RRP44 (DIS3), DNA Methyltransferase 3 Alpha (DNMT3A), Early growth response protein 1 (EGR1), E1A binding protein p300 (EP300), ETS translocation variant 4 (ETV4), Protein FAM46C (FAM46C), Fibroblast growth factor receptor 3 (FGFR3), Far Upstream Element Binding Protein 1 (FUBP1), HIST1H1C, HIST1H1E, HIST1H3G, HIST1H3H, Isocitrate Dehydrogenase 1 (IDH1), Isocitrate Dehydrogenase 2 (IDH2), Insulin-like Growth Factor 1 Receptor (IGF1R), Interferon Regulatory Factor 4 (IRF4), Lysine-Specific Demethylase 5C (KDM5C), Lysine-specific Demethylase 6A (KDM6A), Histone-lysine N-methyltransferase 2A (KMT2A), Lysine Methyltransferase 2B (KMT2B), Lysine Methyltransferase 2C (KMT2C), Lysine Methyltransferase 2D (KMT2D), Kirsten Rat Sarcoma (KRAS), Lymphotoxin-beta (LTB), MAF, MAFB, myc-associated factor X (MAX), Myeloid Differentiation Primary Response Protein (MYD88), Nuclear Receptor Corepressor 1 (NCOR1), Neurofibromin 1 (NF1), Nuclear Factor Of Kappa Light Polypeptide Gene Enhancer In B-Cells Inhibitor (NFKBIA), Neurogenic Locus Notch Homolog Protein 1 (NOTCH1), Neuroblastoma RAS (NRAS), NRM, Phosphatidylinositol-4, 5-Bisphosphate 3-Kinase Catalytic Subunit Alpha (PIK3CA), Protein Phosphatase, Mg2+/Mn2+ Dependent 1D (PPM1D), PRAME Family Member 2 (PRAMEF2), PR Domain Zinc Finger Protein 1 (PRDM1), Serine/threonine-Protein Kinase D2 (PRKD2), Prune Homolog 2 With BCH Domain (PRUNE2), Protein-Tyrosine Phosphatase Non-Receptor Type 11 (PTPN11), RAS P21 Protein Activator 2 (RASA2), Retinoblastoma Associated Protein (RB1), SET Domain Containing 2, Histone Lysine Methyltransferase (SETD2), Splicing Factor 3b Subunit 1 (SF3B1), SP140, Ten Eleven Translocation Methylcytosine Dioxygenase 2 (TET2), TDP-Glucose 4, 6-Dehydratase (TGDS), Tumor Protein p53 (TP53), TNF Receptor Associated Factor 3 (TRAF3), and Zinc Finger Homeobox Protein 3 (ZFHX3).

3. The method of claim 2, wherein the at least one MRD-associated gene comprises KRAS and NRAS.

4. The method of claim 2, wherein the at least one MRD-associated gene comprises TP53 and ATM.

5. The method of claim 2, wherein the at least one MRD-associated gene comprises an MYC oncogene.

6. The method of claim 1, wherein the somatic aberration comprises a single nucleotide variation (SNV), a copy number alteration (CNA), a chromosome translocation breakpoint, or a VDJ rearrangement.

7. The method of claim 1, wherein the sample is obtained from blood, urine, or bone marrow.

8. The method of claim 1, wherein the sample comprises cell free deoxyribonucleic acid (cfDNA) or circulating tumor cells (CTCs).

9. The method of claim 1, wherein the reference sample is obtained from a healthy normal control sample, a MGUS sample, an SMM sample, or an MM sample.

10. The method of claim 1, wherein the somatic aberration of the MM-associated gene is detected via next generation sequencing (NGS), whole exome sequencing (WES), or deep targeted sequencing (DTS).

11. The method of claim 1, further comprising treating the subject with a chemotherapeutic agent, radiation therapy, a corticosteroid, a bone marrow transplant, or a stem cell transplant.

12. The method of claim 11, wherein the chemotherapeutic agent comprises elotuzumab, lenalidomide, dexamethasone, melphlan, vincristine, doxorubicin, etoposide, bendamustine, or cyclophosphamide.

13. The method of claim 1, further comprising repeating the method over time, wherein an increase in somatic alteration of the MM-associated gene over time indicates a corresponding increase in progression of MM.

14. The method of claim 1, wherein the subject is human.

15. A method of determining whether a subject with minimal residual disease (MRD) will relapse to MM in a subject comprising:
obtaining a test sample from a subject having MRD;
detecting a somatic aberration in at least one MM-associated gene in the test sample as compared to the MM-associated gene in a reference sample; and
determining that the subject will relapse to MM.

16. The method of claim 15, further comprising treating the subject with a chemotherapeutic agent, radiation therapy, a corticosteroid, a bone marrow transplant, or a stem cell transplant.

17. The method of claim 15, wherein the sample is obtained from blood, urine, or bone marrow.

18. A method of monitoring therapeutic efficacy of treatment in a subject with MM comprising:
administering treatment to the subject having MM;
obtaining a test sample from the subject;
detecting a somatic aberration in at least one MM-associated gene in the test sample as compared to the MM-associated gene in a reference sample;
determining that the treatment in the subject is not effective if the level of the somatic aberrations in the test sample is higher as compared to the level of somatic aberration in the reference sample, and
modifying treatment of the subject.

19. The method of claim 18, wherein the treatment comprises administration of a chemotherapeutic agent, radiation therapy, corticosteroids, a bone marrow transplant, or a stem cell transplant.

20. The method of claim 18, further comprising repeating the method over time, wherein a decrease in somatic alteration of the MM-associated gene over time indicates that the treatment is effective.
